(19) United States
(12) Patent Application Publication
(10) Pub. No.: US 2024/0141383 A1
(43) Pub. Date: May 2, 2024

(54) VIRAL VECTOR CONSTRUCTS INCORPORATING DNA FOR INHIBITING TOLL LIKE RECEPTORS AND METHODS OF USING THE SAME

(71) Applicant: Kriya Therapeutics, Inc., Palo Alto, CA (US)

(72) Inventors: Nachi GUPTA, San Francisco, CA (US); Michele STONE, Apex, NC (US)

(21) Appl. No.: 18/548,676

(22) PCT Filed: Mar. 4, 2022
(86) PCT No.: PCT/US2022/018986

§ 371 Date: Sep. 1, 2023

Related U.S. Application Data
(60) Provisional application No. 63/156,766, filed on Mar. 4, 2021.

(51) Int. Cl. C12N 15/86; A61P 37/06; C12N 15/117

(52) U.S. Cl. CPC C12N 15/86; A61P 37/06; C12N 15/117; C12N 2310/17; C12N 2750/14143; C12N 2750/14152

(57) ABSTRACT

The present disclosure provides the vector constructs comprising (a) a polynucleotide comprising a promoter operably linked to a nucleic acid of interest; (b) a first terminal repeat and a second terminal repeat; and (c) a backbone polynucleotide comprising a nucleic acid sequence that modulates a Toll-like receptor (TLR). Some aspects of the disclosure are related to methods for packaging the nucleic acid of interest and the nucleic acid sequence that modulates the TLR in an AAV particle, and some aspects are related to methods of modulating an immune response in a subject, comprising administering to said subject an effective amount of such AAV particles.

### CROSS REFERENCE TO RELATED APPLICATIONS

The present application claims the priority benefit of U.S. Provisional Application No. 63/156,766 filed Mar. 4, 2021, which is incorporated herein by reference in its entirety.

### REFERENCE TO SEQUENCE LISTING SUBMITTED ELECTRONICALLY

The content of the electronically submitted sequence listing in ASCII text file (Name: 4525_033PC01_Seqlisting_ST25.txt; Size: 13,368 bytes; and Date of Creation: Mar. 3, 2022) filed with the application is incorporated herein by reference in its entirety.

### BACKGROUND

A variety of physical and chemical methods have been developed for introducing exogenous DNA into eukaryotic cells including viruses, which have generally been shown to be more efficient for this purpose. Several DNA-containing viruses such as parvoviruses, adenoviruses, herpesviruses and poxviruses, and RNA-containing viruses, such as retroviruses, have been used to develop eukaryotic cloning and expression vectors. Some challenges with the viral vectors include low efficiency, packaging capacity, and a lack of specificity.

Another key concern is the inflammatory response that can be elicited by the viral delivery vector. For instance, following AAV administration in mice, artificially induced systemic inflammation, such as the upregulation of tumor necrosis factor (TNF), has been shown to lead to a decline in transgene expression in the liver. Breous, E., et al., Gastroenterology 141(1): 348-57 (2011). Similarly, the use of AAVrh.32.33, which induces higher liver enzymes than other tested AAV serotypes, was shown to lead to a decline in transgene expression to below detection levels in mice. Wang, L., et al., Mol Ther. 18(1): 118-25 (2010). Immunosuppressive and antiinflammatory drugs have been used with viral vector based gene therapy, but these drugs can compromise the patient's immune system during treatment.

Furthermore, viral vectors can contain undesirable contaminants. For example, AAV viral vector preparations often contain contaminating sequences that are packaged alongside the expression cassette at a low rate. These sequences can originate from production plasmid DNA, or chromosomal DNA from producer cell lines. Brimble, et al., “AAV Preparations Contain Contamination from DNA Sequences in Production Plasmids Directly Outside of the ITRs” Molecular Therapy, Vol. 24, Suppl. 1, Abstract 548 (May 2016).

There is a continuing need to improve the efficacy of viral vectors for therapy and for in vivo production of biological products, and in particular to reduce, inhibit, or eliminate unwanted inflammatory responses during gene therapy.

### BRIEF SUMMARY

Certain aspects of the disclosure are directed to a vector construct comprising: (a) a polynucleotide comprising a promoter operably linked to a nucleic acid of interest; (b) a first terminal repeat and a second terminal repeat; and (c) a backbone polynucleotide comprising a nucleic acid sequence that modulates a Toll-like receptor (TLR).

In some aspects, the first and second terminal repeats are inverted terminal repeats (ITRs) or long terminal repeats. In some aspects, the first terminal repeat is adjacent to the 5′ end of the polynucleotide comprising the promoter operably linked to a nucleic acid of interest (e.g., an expression cassette). In some aspects, the second terminal repeat is adjacent to the 3′ end of the polynucleotide comprising the promoter operably linked to a nucleic acid of interest (e.g., an expression cassette). In some aspects, the nucleic acid sequence that modulates the TLR is not between the 5′ and 3′ ITRs.

In some aspects, the backbone polynucleotide comprises two or more copies of the nucleic acid sequence that modulates a TLR. In some aspects, the backbone polynucleotide comprises between 2 to 500 copies, between 2 to 200 copies, between 2 to 150 copies, between 2 to 100 copies, between 2 to 50 copies, between 2 to 40 copies, between 2 to 30 copies, between 2 to 25 copies, between 2 to 20 copies, between, 2 to 15 copies, or between 2 to 10 copies of the nucleic acid sequence that modulates the TLR.

In some aspects, the backbone polynucleotide comprises a linker positioned between the two or more copies of the nucleic acid sequence that modulates the TLR. In some aspects, the linker is 3 to 25 nucleotides in length.

In some aspects, the two or more copies of the nucleic acid sequence that modulates the TLR are positioned in tandem with or without the linker in between.

In some aspects, the nucleic acid sequence that modulates the TLR is positioned within 500 nucleotides or less, 450 nucleotides or less, 400 nucleotides or less, 350 nucleotides or less, 300 nucleotides or less, 250 nucleotides or less, 200 nucleotides or less, 150 nucleotides or less, 100 nucleotides or less, or 50 nucleotides or less from the first terminal repeat or the second terminal repeat.

In some aspects, the TLR comprise a TLR3, a TLR4, a TLR7, a TLR8, a TLR9, or any combination thereof. In some aspects, the TLR comprises TLR9.

In some aspects, the backbone polynucleotide comprises two or more copies of the nucleic acid sequences that modulates the TLR, e.g., TLR9.

In some aspects, the nucleic acid sequence that modulates the TLR comprises a sequence selected from any of SEQ ID NOs: 1-28, 29-38, 54, or 55, or any combination thereof. In some aspects, the first and/or second terminal repeat comprises a sequence selected from any one of SEQ ID Nos: 29-38.

In some aspects, the backbone polynucleotide is at least 4000 nucleic acids in length. In some aspects, the backbone polynucleotide is about 4000 to about 8000 nucleic acids in length or about 5000 to about 7000 nucleic acids in length.

In some aspects, the nucleic acid sequence that modulates the TLR comprises about 0.5% to about 10%, about 0.5% to about 5%, about 0.5% to about 4%, about 0.5% to about 3%, about 0.5% to about 2%, or about 0.5% to about 1% of the total nucleic acid sequence of the backbone polynucleotide of the vector construct.

In some aspects, the nucleic acid sequence that modulates the TLR is capable of inhibiting a TLR inflammatory response. In some aspects, the nucleic acid sequence that modulates the TLR is a TLR9 antagonist.

In some aspects, the nucleic acid sequence that modulates the TLR is capable of activating an inflammatory response. In some aspects, the nucleic acid sequence that modulates the TLR is a TLR9 agonist.

In some aspects, the first terminal repeat is an ITR comprising about 75 to about 175 nucleotides in length. In some aspects, the second terminal repeat is an ITR comprising about 75 to about 175 nucleotides in length.

In some aspects, the first terminal repeat is an ITR and/or the second terminal repeat is an ITR from an AAV serotype selected from AAV1, AAV2, AAV3a, AAV3b, AAV4, AAV5, AAV6, AAV7, AAV8, AAV9, AAV10, AAV11, or AAV13, or any functional fragment thereof.

Certain aspects of the disclosure are directed to a method for packaging the nucleic acid of interest and the nucleic acid sequence that modulates the TLR in an AAV capsid, comprising transfecting a cell in vitro with (i) a vector construct disclosed herein and (ii) AAV packaging genes (e.g., one or more plasmids comprising Rep/Cap genes and adenovirus genes), wherein the nucleic acid of interest and the nucleic acid sequence that modulates the TLR (e.g., as part of a partial backbone sequence) are packaged in the AAV capsid.

In some aspects, the AAV capsid is an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

Certain aspects of the disclosure are directed to an AAV particle produced by a method of the disclosure.

Certain aspects of the disclosure are directed to compositions comprising a vector construct, a viral vector, or an AAV particle of the disclosure.

Certain aspects of the disclosure are directed to a method of modulating an immune response in a subject, comprising administering to said subject an effective amount of an viral vector (e.g., AAV vector), an AAV particle, or a composition of the disclosure.

In some aspects, the nucleic acid sequence that modulates the TLR is capable of inhibiting or reducing a TLR inflammatory response.

In some aspects, the method reduces the subject's immune response to a gene therapy (e.g., comprising a viral vector, an AAV particle or a composition of the disclosure). In some aspects, the method reduces the subject's immune response to an AAV gene therapy.

In some aspects, the nucleic acid sequence that modulates the TLR is capable of activating or increasing an inflammatory response in a subject. In some aspects, the method disclosed herein enhances the subject's immune response to a tumor.

Certain aspects of the disclosure are directed to a method of reducing immunogenicity of an AAV particle comprising packaging a portion of a backbone polynucleotide comprising a nucleic acid sequence that modulates a Toll-like receptor (TLR) into an AAV capsid thereby producing the AAV particle, whereby the AAV particle has a reduced inflammatory response in a host as compared to an AAV particle that does not comprise the portion of a backbone comprising the nucleic acid sequence that modulates the TLR.

Certain aspects of the disclosure are directed to a method of enhancing immunogenicity of an AAV particle comprising packaging a portion of a backbone polynucleotide comprising a nucleic acid sequence that modulates a Toll-like receptor (TLR) into an AAV capsid thereby producing the AAV particle, whereby the AAV particle causes an enhanced inflammatory response in a host as compared to an AAV particle that does not comprise the portion of a backbone comprising the nucleic acid sequence that modulates the TLR.

In some aspects, the AAV capsid is an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

In some aspects, the TLR comprise a TLR3, a TLR4, a TLR7, a TLR8, a TLR9, or any combination thereof. In some aspects, the TLR comprises TLR9.

In some aspects, the backbone polynucleotide comprises two or more copies of the nucleic acid sequences that modulates a TLR, e.g., TLR9. In some aspects, the nucleic acid sequences that modulates a TLR, e.g., TLR9, comprise tandem repeats.

In some aspects, the nucleic acid sequence that modulates the TLR comprises a sequence selected from any of SEQ ID NOs: 1-28, 29-38, 54 or 55, or any combination thereof. In some aspects, the first and/or second terminal repeat comprises a sequence selected from any one of SEQ ID Nos: 29-38.

In some aspects, the nucleic acid sequence that modulates TLR comprises between 2 to 20 or 4 to 18 (e.g., 2, 3, 4, 5, 6, 7, 8, 9, 10, 11, 12, 13, 14, 15, 16, 17, 18, 19, or 20) tandem repeats of TTAGGG (SEQ ID NO: 55). In some aspects, the nucleic acid sequence that modulates TLR comprises a sequence of SEQ ID NO: 3.

In some aspects, wherein the polynucleotide comprising the promoter operably linked to a nucleic acid of interest (e.g., an expression cassette) is located between the first and second terminal repeat and measures in length less than a viral genome.

In some aspects, the polynucleotide comprising the promoter operably linked to a nucleic acid of interest measures in length less than a single-stranded AAV viral genome.

In some aspects, the polynucleotide comprising the promoter operably linked to a nucleic acid of interest measure in length less than 4700 nucleotides.

In some aspects, the polynucleotide comprising the promoter operably linked to a nucleic acid of interest measures in length less than a self-complementary AAV genome.

In some aspects, the polynucleotide comprising the promoter operably linked to a nucleic acid of interest is self-complementary and measures in length less than 2300 nucleotides.

In some aspects, the polynucleotide comprising the promoter operably linked to the nucleic acid of interest and the 5′ and 3′ ITRs (e.g., an expression cassette) measures in length less than an AAV genome (e.g. less than 4.7 kb) and the nucleic acid sequence that modulates a TLR (e.g., TLR9) is positioned within 500 nucleotides or less, 450 nucleotides or less, 400 nucleotides or less, 350 nucleotides or less, 300 nucleotides or less, 250 nucleotides or less, 200 nucleotides or less, 150 nucleotides or less, 100 nucleotides or less, or 50 nucleotides or less from the first terminal repeat or the second terminal repeat. In some aspects, the first terminal repeat is adjacent to the 5′ end of the polynucleotide comprising the promoter operably linked to a nucleic acid of interest (e.g., an expression cassette). In some aspects, the second terminal repeat is adjacent to the 3′ end of the polynucleotide comprising the promoter operably linked to a nucleic acid of interest (e.g., an expression cassette).

In some aspects, the polynucleotide comprising the promoter operably linked to a nucleic acid of interest measures in length less than an AAV genomes and the nucleic acid sequence that modulates a TLR (e.g., TLR9) is adjacent to a first or a second inverted repeat. In some aspects, the nucleic acid sequence that modulates the TLR is not between the 5′ and 3′ ITRs.

In some aspects, the backbone polynucleotide of the vector construct comprises between 2 to 500 copies, between 2 to 200 copies, between 2 to 150 copies, between 2 to 100 copies, between 2 to 50 copies, between 2 to 40 copies, between 2 to 30 copies, between 2 to 25 copies, between 2 to 20 copies, between, 2 to 15 copies, or between 2 to 10 copies of the nucleic acid sequence that modulates the TLR (e.g., TLR9).

In some aspects, the AAV particle comprises about 0.01% to about 2% of the polynucleotide backbone sequence from the vector construct.

### BRIEF DESCRIPTION OF THE DRAWINGS

FIG. 1 shows a schematic of an exemplary vector construct designed to include a backbone, flanking terminal repeats (e.g., ITRs), a promoter, an open reading frame (ORF), and polyA.

FIG. 2 shows a schematic example of a vector construct for production of recombinant virions (e.g., AAV virions). (1) represents an open reading frame (ORF), (2) represent terminal repeats (e.g., ITRs), and (3) represents the backbone. (A) shows packaging of a nucleic acid sequences including the ORF between two flanking terminal repeats (e.g., ITRs) without backbone sequence packaged into recombinant virions. (B) shows examples of packaging of a nucleic acid sequence including the ORF between two flanking terminal repeats (e.g., ITRs) including partial backbone sequence packaged into recombinant virions. (C) shows examples of flanking terminal repeats (e.g., ITRs) including partial backbone sequence packaged into recombinant virions. (D) shows examples of backbone DNA fragments packaged into recombinant virions.

FIG. 3A shows a schematic example of a vector construct including nucleic acid sequence(s) that modulates a Toll-like receptor (TLR) (shown as X symbols) for production of recombinant virions (e.g., AAV virions). (1) represents an open reading frame (ORF), (2) represent terminal repeats (e.g., ITRs), (3) represents the backbone, and (4, shown as an X) represents a nucleic acid sequence that modulates a Toll-like receptor (TLR). (A) shows packaging of a nucleic acid sequences including the ORF between two flanking terminal repeats (e.g., ITRs) without backbone sequence packaged into recombinant virions. (B) shows examples of packaging of a nucleic acid sequence including the ORF between two flanking terminal repeats (e.g., ITRs) including partial backbone sequence including a nucleic acid sequence that modulates a TLR packaged into recombinant virions. (C) shows examples of flanking terminal repeats (e.g., ITRs) including partial backbone sequence including a nucleic acid sequence that modulates a TLR packaged into recombinant virions. (D) shows examples of backbone DNA fragments including a nucleic acid sequence that modulates a TLR packaged into recombinant virions.

FIG. 3B shows a schematic example of a vector construct similar to the description in FIG. 3A, but also illustrates alternative locations in variation 4 of the nucleic acid sequences that modulates a Toll-like receptor (TLR) (shown as an X) outside the ITRs. (B) shows examples of packaging of a nucleic acid sequence including the ORF between two flanking terminal repeats (e.g., ITRs) including partial backbone sequence including a nucleic acid sequence that modulates a TLR packaged into recombinant virions, where the nucleic acid sequence that modulates the TLR can be derived from the backbone sequence(s) 5′, 3′, or outside both ends of ITRs and thereafter packaged into recombinant virions.

FIG. 4 shows NF-κB/AP-1-induced secretion of secreted embryonic alkaline phosphatase (SEAP) following incubation of HEK-Dual™ TLR9 reporter cells with oligonucleotides: a 5′ TLR9-stimulatory ODN2006 sequence combined with a 3′ ODN sequence having four copies of TTAGGG (SEQ ID NO: 3) (ODN2006-TTAGGG, SEQ ID NO: 50) or a 5′ TLR9-stimulatory ODN2006 sequence combined with a 3′ sequence having four copies of TTCGCG (SEQ ID NO: 54) (ODN2006-TTCGCG, SEQ ID NO: 51). Control oligonucleotides included either no TLR-stimulatory sequences (Control-Control, SEQ ID NO: 53) or a 5′ TLR9-stimulatory ODN2006 sequence combined with a 3′ control sequence (ODN2006-Control, SEQ ID NO: 52).

FIG. 5A shows a schematic of an exemplary vector construct designed to include a backbone, flanking terminal repeats (e.g., ITRs), a promoter, an open reading frame (ORF), and polyA. The backbone in FIG. 5A is modified to include repeats of TTAGGG (SEQ ID NO: 55) in opposite orientations outside of the 5′ and the 3′ ends of the ITRs.

FIG. 5B shows a schematic of an exemplary vector construct similar to the vector in FIG. 5A except without the repeats in the backbone.

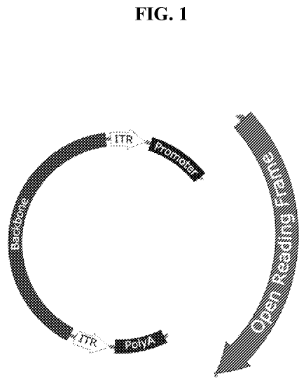

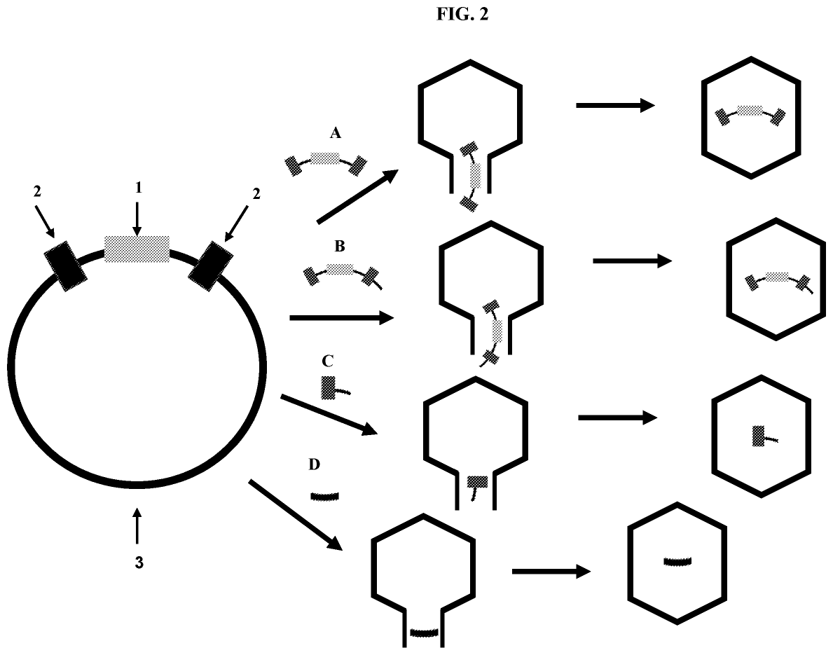

### DETAILED DESCRIPTION OF THE INVENTION

The present disclosure is directed to compositions and methods comprising improved vector constructs, which are designed to modulate (e.g., reduce the risk of) an inflammatory response in subjects administered gene therapy using a viral vector. Some aspects of the present disclosure relate to a vector construct comprising: (a) a polynucleotide comprising a promoter operably linked to a nucleic acid of interest (e.g., an expression cassette); (b) a first terminal repeat and a second terminal repeat (e.g., flanking the expression cassette); and (c) a backbone polynucleotide comprising a nucleic acid sequence that modulates a Toll-like receptor (TLR). In some aspects, the first and second terminal repeats are inverted terminal repeats (ITRs) or long terminal repeats. In some aspects, the nucleic acid sequence that modulates TLR is capable of inhibiting an inflammatory response. In some aspects, the nucleic acid that modulates TLR is capable of activating an inflammatory response. In some aspects, the TLR comprises a TLR1, a TLR2, TLR3, TLR4, TLR5, TLR6, TLR7, TLR8, TLR9, TLR10, or any combination thereof. In some aspects, the TLR comprises a TLR3, a TLR4, a TLR7, a TLR8, a TLR9, or any combination thereof. In some aspects, the TLR comprises TLR9. In some aspects, the nucleic acid sequence that modulates TLR is an agonist or an antagonist of TRL9. Some aspects of the present disclosure generally relate to a method for packaging the nucleic acid of interest and the nucleic acid sequence that modulates the TLR in an AAV capsid, comprising transfecting a cell in vitro with (i) a vector construct comprising (a) a polynucleotide of interest (e.g., encoding a therapeutic protein) and (b) one or more nucleic acid sequences that modulate a TLR and (ii) one or more AAV packaging genes (e.g., one or more plasmids comprising Rep/Cap genes and adenovirus genes), wherein the nucleic acid of interest and the nucleic acid sequence that modulates the TLR are packaged in the AAV capsid. In some instances, the AAV capsid encapsulates a sequence selected from any of SEQ ID NOs: 1-38, 54, 55, or any combination thereof, and a nucleic acid of interest.

Some aspects of the present disclosure generally relate to a method of modulating an immune response in a subject, comprising administering to said subject an effective amount of the AAV particle as described herein, e.g., an AAV particle comprising a polynucleotide of interest and further comprising a nucleic acid sequence that modulates a TLR (e.g., derived from a portion or a fragment of the vector construct backbone). In some aspects, the method reduces the subject's immune response to a gene therapy, e.g., by reducing the subject's inflammatory response to a gene therapy comprising administration of the AAV particle.

Some aspects of the present disclosure generally relate to a method of reducing immunogenicity of an AAV particle comprising packaging a portion (or a fragment) of a backbone polynucleotide comprising a nucleic acid sequence that modulates a Toll-like receptor (TLR) into an AAV capsid, whereby the resulting AAV particle has a reduced inflammatory response in a host as compared to an AAV particle that does not comprise the portion (or the fragment) of the backbone comprising the nucleic acid sequence that modulates the TLR.

Some aspects of the present disclosure generally relate to a method of enhancing immunogenicity of an AAV particle comprising packaging a portion (or a fragment) of a backbone polynucleotide comprising a nucleic acid sequence that modulates a Toll-like receptor (TLR) into an AAV capsid, whereby the resulting AAV particle has an enhanced inflammatory response in a host as compared to an AAV particle that does not comprise the portion (or fragment) of the backbone comprising the nucleic acid sequence that modulates the TLR.

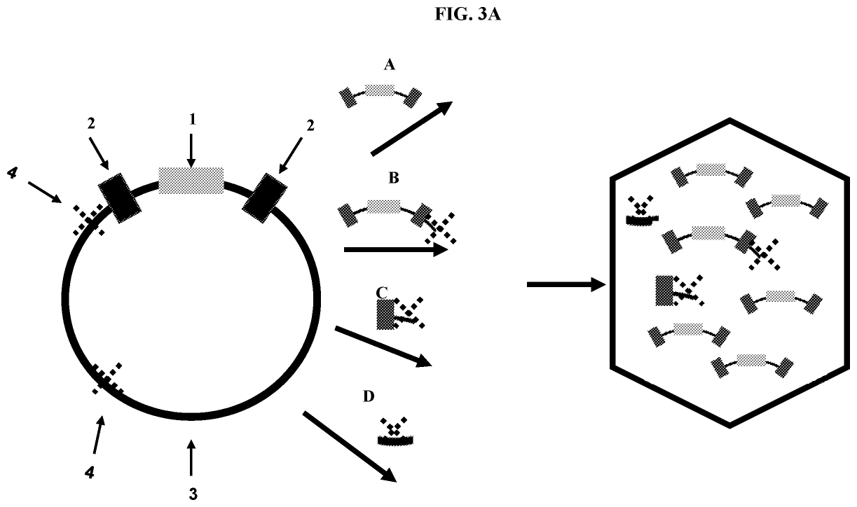

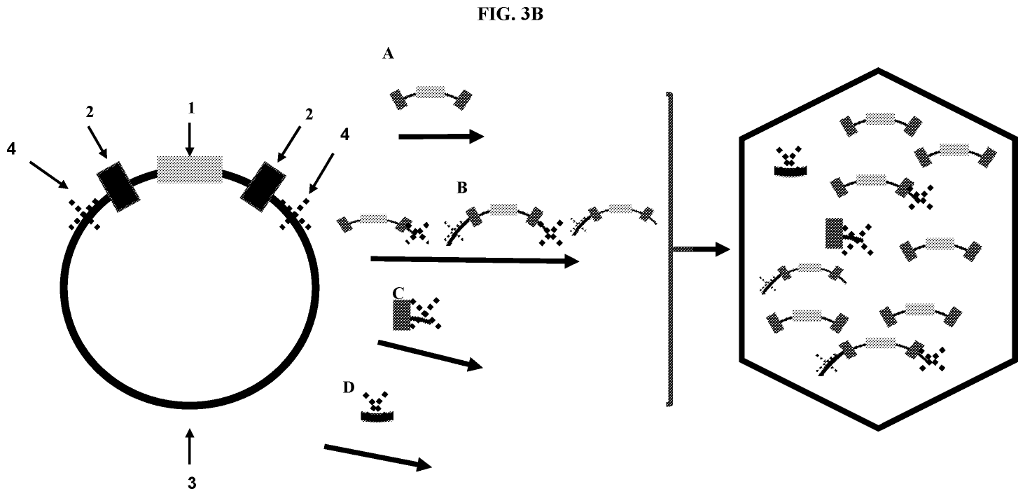

In some aspects, the portion or fragment of the backbone comprising a nucleic acid sequence that modulates a TLR can be linked to a terminal repeat (e.g., ITR) sequence (e.g., see FIG. 3A or 3B, examples B and C) or can be a portion or fragment of the backbone that does not comprise a terminal repeat (e.g., ITR) sequence (e.g., see FIG. 3A or 3B, examples D).

### I. Definitions

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower) unless stated otherwise herein.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

The term “expression vector or construct” means any type of genetic construct containing a nucleic acid in which part or all of the nucleic acid encoding sequence is capable of being transcribed.

As used herein, the term “delivery vector” or “vector” refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “delivery vector” or “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. In some aspects, insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, vectors are contemplated to be those vectors in which the nucleic acid segment to be transcribed is positioned under the transcriptional control of a promoter. In some aspects, the delivery vector is selected from the group consisting of a viral vector (e.g., an AAV vector), a plasmid, a lipid, a protein particle, a bacterial vector, and a lysosome. Some aspects of the disclosure are directed to biological vectors, which can include viruses, particularly attenuated and/or replication-deficient viruses.

A “viral vector” can include a sequence that comprises one or more polynucleotide regions encoding or comprising a molecule of interest, e.g., a protein, a peptide, and an oligonucleotide or a plurality thereof. Viral vectors can be used to deliver genetic materials into cells. Viral vectors can be modified for specific applications. In some aspects, the delivery vector of the disclosure is a viral vector selected from the group consisting of an adeno-associated viral (AAV) vector, an adenoviral vector, a lentiviral vector, or a retroviral vector.

The term “adeno-associated virus vector” or “AAV vector” as used herein refers to any vector that comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, bovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome” and “AAV vector” can be used interchangeably. In some aspects, the AAV vector is modified or mutated relative to the wild-type AAV serotype sequence.

As used herein, the term “AAV particle” or “AAV virion” are used interchangeably and generally refer to an AAV virus that comprises an AAV capsid encapsulating an AAV vector having at least one payload region (e.g., a polynucleotide of interest) and at least one inverted terminal repeat (ITR) region. In some aspects, the AAV particles of the present disclosure further comprise a polynucleotide that modulates a TLR, e.g., as part of an AAV vector backbone sequence that is encapsulated in an AAV capsid.

As used herein, the term “promoter” refers to a DNA sequence recognized by the synthetic machinery of the cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible by external signals or agents; such elements can be located in the 5′ or 3′ regions of the native gene. In some aspects, the promoter is a constitutively active promoter, a cell-type specific promoter, or an inducible promoter.

As used herein, the term “regulatable promoter” is any promoter whose activity is affected by a cis or trans acting factor (e.g., an inducible promoter, such as an external signal or agent).

As used herein, the term “constitutive promoter” is any promoter that directs RNA production in many or all tissue/cell types at most times, e.g., the human CMV immediate early enhancer/promoter region that promotes constitutive expression of cloned DNA inserts in mammalian cells.

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

The terms “transcriptional regulatory protein,” “transcriptional regulatory factor,” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA can be indirect by way of binding to another protein that in turn binds to, or is bound to a DNA response element.

As used herein, the term “termination signal sequence” can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

As used herein, the term “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). “Under translational control of an IRES” as used herein means that translation is associated with the IRES and proceeds in a cap-independent manner.

The term “self-processing cleavage site” or “self-processing cleavage sequence,” as used herein refers to a post-translational or co-translational processing cleavage site or sequence. Such a “self-processing cleavage” site or sequence refers to a DNA or amino acid sequence, exemplified herein by a 2A site, sequence or domain or a 2A-like site, sequence or domain. The term “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

As used herein, the term “additional proteolytic cleavage site,” refers to a sequence that is incorporated into an expression construct of the disclosure adjacent a self-processing cleavage site, such as a 2A or 2A like sequence, and provides a means to remove additional amino acids that remain following cleavage by the self-processing cleavage sequence. Exemplary “additional proteolytic cleavage sites” are described herein and include, but are not limited to, furin cleavage sites with the consensus sequence RXK(R)R (SEQ ID NO: 47). Such furin cleavage sites can be cleaved by endogenous subtilisin-like proteases, such as furin and other serine proteases within the protein secretion pathway. In some aspects, other exemplary “additional proteolytic cleavage sites” can be used, as described in e.g., Lie et al., Sci Rep 7, 2193 (2017).

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene and/or the molecule encoded by a nucleic acid of interest. The term “operably linked” means that a DNA sequence and a regulatory sequence(s) are connected in such a way as to permit gene expression and/or expression of the molecule encoded by a nucleic acid of interest when the appropriate molecules (e.g., transcriptional activator proteins) are bound to the regulatory sequence(s). The term “operably inserted” means that the nucleic acid of interest, e.g., a DNA sequence, introduced into the cell is positioned adjacent a DNA sequence which directs transcription and translation of the introduced DNA (i.e., facilitates the production of, e.g., a polypeptide encoded by a nucleic acid of interest).

A “coding sequence” or a sequence “encoding” a particular molecule (e.g., a protein, e.g., an antibody or antigen-binding fragment thereof) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., an AAV vector) that is derived from a second nucleic acid sequence (e.g., another AAV vector) can include a nucleotide sequence that is identical or substantially similar to the nucleotide sequence of the second nucleic acid sequence.

In some aspects of the polynucleotides described herein, the derived species can be obtained by, for example, naturally occurring mutagenesis, artificial directed mutagenesis or artificial random mutagenesis. The mutagenesis used to derive polynucleotides can be intentionally directed or intentionally random, or a mixture of each. The mutagenesis of a polynucleotide to create a different polynucleotide derived from the first can be a random event (e.g., caused by polymerase infidelity) and the identification of the derived polynucleotide can be made by appropriate screening methods.

As used herein, the term “mutation” refers to any changing of the structure of a gene, resulting in a variant (also called “mutant”) form that can be transmitted to subsequent generations. Mutations in a gene can be caused by the alternation of single base in DNA, or the deletion, insertion, or rearrangement of larger sections of genes or chromosomes.

As used herein, the term “administration” refers to the administration of a composition of the present disclosure (e.g., a viral vector (e.g., AAV vector), an AAV particle, or the gene therapy composition disclosed herein) to a subject or system. Administration to an animal subject (e.g., to a human) can be by any appropriate route, such as intramuscular, intravenous, or to a secretory organ.

As used herein, the term “modified” refers to a changed state or structure of a molecule of the disclosure. Molecules can be modified in many ways including chemically, structurally, and functionally.

As used herein, the term “synthetic” means produced, prepared, and/or

manufactured by the hand of man. Synthesis of polynucleotides or polypeptides or other molecules of the present disclosure can be chemical or enzymatic.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA. The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers.

Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

The term “mRNA,” as used herein, refers to a single stranded RNA that encodes the amino acid sequence of one or more polypeptide chains.

The term “antisense,” as used herein, refers to a nucleic acid that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides can hybridize to each other even if they are not completely complementary to each other, provided that each has at least one region that is substantially complementary to the other.

The terms “antisense strand” and “guide strand” refer to the strand of a dsRNA, e.g., an shRNA, that includes a region that is substantially complementary to a target sequence, e.g., mRNA. The antisense strand has sequence sufficiently complementary to the desired target mRNA sequence to direct target-specific silencing, e.g., complementarity sufficient to trigger the destruction of the desired target mRNA by the RNAi machinery or process.

The terms “sense strand” and “passenger strand,” as used herein, refer to the strand of a dsRNA, e.g., an shRNA, that includes a region that is substantially complementary to a region of the antisense strand as that term is defined herein. The antisense and sense strands of a dsRNA, e.g., an shRNA, are hybridized to form a duplex structure.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

The phrase “contacting a cell” (e.g., contacting a cell with a vector construct, an viral vector, an AAV particle, or the gene therapy composition of the present disclosure) as used herein, includes contacting a cell directly or indirectly. In some aspects, contacting a cell with vector construct, a viral vector (e.g., AAV vector), an AAV particle, or the gene therapy composition includes contacting a cell in vitro with the vector construct, the viral vector (e.g., AAV vector), the AAV particle, or the gene therapy composition or contacting a cell in vivo with the vector construct, the viral vector (e.g., AAV vector), the AAV particle, or the gene therapy composition. Thus, for example, the vector construct, the viral vector (e.g., AAV vector), the AAV particle, or the gene therapy composition can be put into physical contact with the cell by the individual performing the method, or alternatively, the vector construct, the viral vector (e.g., AAV vector), the AAV particle, or the gene therapy composition can be put into a situation that will permit or cause it to subsequently come into contact with the cell. In some aspects, contacting a cell in vitro can be done, for example, by incubating the cell with the vector construct, the viral vector (e.g., AAV vector), the AAV particle, or the gene therapy composition. In some aspects, contacting a cell in vivo can be done, for example, by injecting the vector construct, the viral vector (e.g., AAV vector), the AAV particle, or the gene therapy composition of the disclosure into or near the tissue where the cell is located (e.g., a secretory organ), or by injecting the vector construct, the viral vector (e.g., AAV vector), the AAV particle, or the gene therapy composition into another area, e.g., the bloodstream or the subcutaneous space, such that the agent will subsequently reach the tissue where the cell to be contacted is located. For example, the vector construct, the viral vector (e.g., AAV vector), or the AAV particle can be encapsulated and/or coupled to a ligand that directs the vector construct, the viral vector (e.g., AAV vector), or the AAV particle to a site of interest. Combinations of in vitro and in vivo methods of contacting are also possible. For example, a cell can be contacted in vitro with a vector construct, an viral vector (e.g., AAV vector), an AAV particle, or the gene therapy composition and subsequently transplanted into a subject.

In some aspects, contacting a cell with a vector construct, a viral vector (e.g., AAV vector), an AAV particle, or a gene therapy composition of the present disclosure includes “introducing” or “delivering” (directly or indirectly) the vector construct, the viral vector (e.g., AAV vector), the AAV particle, or the gene therapy composition into the cell by facilitating or effecting uptake or absorption into the cell. Introducing a vector construct, a viral vector (e.g., AAV vector), an AAV particle, or a gene therapy composition into a cell can be in vitro and/or in vivo. For example, for in vivo introduction, the vector construct, the viral vector (e.g., AAV vector), the AAV particle, or the gene therapy composition can be injected into a specific tissue site (e.g., the locus where a therapeutic effect is desired) or administered systemically (e.g., administering a AAV vector targeted to a locus where a therapeutic effect is desired). In vitro introduction into a cell includes methods known in the art such as electroporation and lipofection.

As used herein, the terms “effective amount,” “therapeutically effective amount,” and a “sufficient amount” of, e.g., a vector construct, a viral vector (e.g., AAV vector), an AAV particle, or a gene therapy composition disclosed herein refer to a quantity sufficient to, when administered to the subject, including a human, effect beneficial or desired results, including clinical results, and, as such, an “effective amount” or synonym thereto depends on the context in which it is being applied. In some aspects, a therapeutically effective amount of an agent (e.g., an viral vector (e.g., AAV vector), an AAV particle, the gene therapy composition disclosed herein) is an amount that results in a beneficial or desired result in a subject as compared to a control.

The amount of a given agent (e.g., an viral vector (e.g., AAV vector), an AAV

particle, or the gene therapy composition disclosed herein) will correspond to such an amount will vary depending upon various factors, such as the given agent, the pharmaceutical formulation, the route of administration, the type of disease or disorder, the identity of the subject (e.g., age, sex, and/or weight) or host being treated, and the like.

As used herein, the term “gene therapy” is the insertion of nucleic acid sequences (e.g., a nucleic acid comprising a promoter operably linked to a polynucleotide of interest, such as one encoding a therapeutic molecule) into an individual's cells and/or tissues to treat, reduce the symptoms of, or reduce the likelihood of a disease. Gene therapy also includes insertion of transgene that are inhibitory in nature, i.e., that inhibit, decrease or reduce expression, activity or function of an endogenous gene or protein, such as an undesirable or aberrant (e.g., pathogenic) gene or protein. Such transgenes can be exogenous. An exogenous molecule or sequence is understood to be molecule or sequence not normally occurring in the cell, tissue and/or individual to be treated. Both acquired and congenital diseases are amenable to gene therapy.

The term “prophylactically effective amount,” as used herein, includes the amount of an agent, (e.g., an viral vector (e.g., AAV vector), an AAV particle, or the gene therapy composition disclosed herein) that, when administered to a subject having or predisposed to have a disease or infection is sufficient to prevent, reduce the symptoms of, or ameliorate the disease or infection or one or more symptoms of the disease or infection. Ameliorating the disease or infection includes slowing the course of the disease or infection or reducing the severity of later-developing disease or infection. The “prophylactically effective amount” can vary depending on the characteristics of the agent, e.g., an viral vector (e.g., AAV vector), an AAV particle, or the gene therapy composition, how the agent is administered, the degree of risk of disease, and the history, age, weight, family history, genetic makeup, the types of preceding or concomitant treatments, if any, and other individual characteristics of the patient to be treated.

As used herein, “off target” refers to any unintended effect on any one or more target, gene, or cellular transcript.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal (e.g., human subject), plant, or microbe or cell or tissue thereof).

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large, e.g., DNA encoding one or more genes and organized into an expression plasmid, e.g., a vector.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (MA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

By “level” is meant a level or activity of a protein, or mRNA encoding the protein, optionally as compared to a reference. The reference can be any useful reference, as defined herein. By a “decreased level” or an “increased level” of a protein is meant a decrease or increase in protein level, as compared to a reference.

A level of a protein can be expressed in mass/vol (e.g., g/dL, mg/mL, μg/mL, ng/mL) or percentage relative to total protein or mRNA in a sample.

The term “pharmaceutical composition,” as used herein, represents a composition comprising a compound or molecule described herein, e.g., a viral vector (e.g., AAV vector) or AAV particle, formulated with a pharmaceutically acceptable excipient. In some aspects, the pharmaceutical composition can be manufactured or sold with the approval of a governmental regulatory agency as part of a therapeutic regimen for the treatment of disease in a mammal.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration.

As used herein, the term “subject” refers to any organism to which a composition disclosed herein, e.g., a construct of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. In some aspects, treating reduces or lessens the symptoms associated with a disease or disorder. In some aspects, the treating results in a beneficial or desired clinical result. In some aspects, the disease is an infectious disease.

Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years.

As used herein, the terms “nucleic acid of interest”, “polynucleotide of interest”, and the like, generally refer any one or more nucleic acid sequences that encode one or more corresponding molecules, e.g., proteins, whose expression is desired, e.g., for gene therapy. In some aspects, the nucleic acid of interest are selected for placement into a construct and/or delivery vector, e.g., a vector construct, e.g., a viral vector construct as described herein. In some aspects, the nucleic acid of interest can be any gene sequence or functional portion thereof from any organism.

As used herein, the term “terminal repeat” refers to a nucleotide sequences repeated on both the 5′ and 3′ ends of a sequence that comprises a coding sequence. For example, the hallmarks of a transposon are that it is flanked by inverted repeats on each end and the inverted repeats are flanked by direct repeats.

As used herein, the term “long terminal repeat” generally refers to a pair of sequences of DNA (e.g., identical sequences), typically several hundred base pairs long, which can occur in eukaryotic genomes on either end of a series of genes or pseudogenes that form a retrotransposon or an endogenous retrovirus or a retroviral provirus. Generally, retroviral genomes are flanked by LTRs, while there are some retrotransposons without LTRs. In some instances, an element flanked by a pair of LTRs encodes a reverse transcriptase and an integrase, allowing the element to be copied and inserted at a different location of the genome.

As used herein, the term “inverted terminal repeat” or “ITR” generally refers to a single stranded sequence of nucleotides that is followed downstream by its reverse complement. ITRs can occur at the boundaries in transposons. In some aspects, the intervening sequence of nucleotides between the initial sequence and the reverse complement can be any length, including zero. For example, 5′---TTACGnnnnnnCGTAA---3′ (SEQ ID NO: 49) is an inverted repeat sequence. ITRs can be positioned at the tips of the transposon that signal where breakage and joining should occur. In some instances, ITRs comprise one or more binding sites for a transposase. In some instances, a binding site for a host factor important for recombination can also be part of an ITR. The ITRs can function as origins of replication comprising recognition sites for replication. ITRs comprise sequence regions, which can be complementary and symmetrically arranged. ITRs can be incorporated into vector constructs of the disclosure and can be comprised of naturally occurring polynucleotide sequences or recombinantly derived polynucleotide sequences. In some instances, ITRs are serve as origins of replication and as packaging signals for the viral genome.

As used herein, the terms “backbone” and “backbone polynucleotide” refers to a polynucleotide sequence of a polynucleotide-based vector or plasmid, which does not include the transgene, regulatory elements for the transgene, or terminal repeat sequences.

As used herein, a backbone does not include a promoter, an open reading frame comprising a polynucleotide of interest, a polyA tail, or terminal repeat sequences. Backbone polynucleotides can be engineered to encode selectable markers or reporters that provide for the selection or identification of cells that have incorporated the polynucleotide-based vector. Expression of selectable markers or reporters allows identification and/or selection of host cells that incorporate and express other coding regions contained on the polynucleotide-based vector.

As used herein, the term “modulate” generally refers to any change, alteration, adjustment, and/or regulation of a given aspect of a process. For example, modulation can comprise an increase or decrease of any degree of a signaling event, an increase or a decrease of any degree of a level of expression of a protein of interest, and the like. In some aspects, the viral vector constructs disclosed herein modulate an inflammatory response associated with a given toll-like receptor (TLR), e.g., by increasing or decreasing the inflammatory response associated with a given TLR.

As used herein, the terms “CpG oligodeoxynucleotide” or “CpG ODN” refer to short DNA sequences that contain a cytosine triphosphate deoxynucleotide (“C”) followed by a guanine triphosphate deoxynucleotide (“G”), where the “p” refers to the phosphodiester link between consecutive nucleotides. In some aspects, ODNs can have a modified phosphorothioate (PS) backbone instead. In some aspects, CpG ODNs are recognized by one or more TLRs, e.g., TLR9. In some aspects, the CpG ODNs are recognized by multiple TLRs, e.g., TLR7, TLR8, and TLR9. CpG ODNs can have immunologic effects, such as stimulating monocytes, macrophages, and dendritic cells that then produce several cytokines, including the TH1 cytokine interleukin 12. In some aspects, this cytokine-producing effect can synergize with CpG ODN to induce NK cell production of interferon γ. In some aspects, CpG ODNs can trigger the production of reactive oxygen species which can activate NF-κB, and this activation, in turn, can lead to cellular activation of various cellular processes. In some aspects, CpG ODNs can enhance antibody-dependent cellular cytotoxicity and improve the in vivo efficacy of monoclonal antibody therapy, e.g., as was shown in a syngeneic murine lymphoma model (Wooldridge, J. E., et al., Blood 89: 2994-2998 (1997)). In some aspects, CpG ODNs can induce activation of antigen-presenting cells and enhance production of cytokines known to participate in the development of an active immune response. In some aspects, CpG ODNs can enhance B cell activation.

As used herein the terms “ODN” or “oligonucleotide” and “oligo” refer to short DNA sequences.

As used herein, the terms “linker”, “spacer” “linker region”, and “spacer region” refer to one or more nucleotides or amino acids that reside between two regions and/or domains of a construct or functional element, such that the two regions and/or domains of the construct are separated and/or connected by the linker. In some aspects, the backbone polynucleotide comprises a linker positioned between the two or more nucleic acid sequences that modulates the TLR. In some aspects, the linker comprises one or more nucleotides. In some aspects, the linker is 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more nucleotides in length. In some aspects, the linker comprises DNA and/or RNA bases. In some aspects, the linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some aspects, the linker is 1 to 30, 1 to 29, 1 to 28, 1 to 27, 1 to 26, 1 to 25, 1 to 24, 1 to 23, 1 to 22, 1 to 21, 1 to 20, 2 to 30, 2 to 29, 2 to 28, 2 to 27, 2 to 26, 2 to 25, 2 to 24, 2 to 23, 2 to 22, 2 to 21, 2 to 20, 3 to 30, 3 to 29, 3 to 28, 3 to 27, 3 to 26, 3 to 25, 3 to 24, 3 to 23, 3 to 22, 3-21, or 3-20 nucleotides in length. In some aspects, the linker is 3 to 25 nucleotides in length. In some aspects, the linker comprises a sequence selected from the sequences of Table 3. In some aspects, the linker comprises a sequence selected from the group consisting of SEQ ID NOs: 39-46, or any combination thereof.

As used herein, the term “positioned” is given its general meaning in the art and generally refers to putting or arranging elements in particular place or way. For example, a nucleic acid sequence can be positioned upstream, downstream, 5′ to, 3′ to, or between one or more other nucleic acid sequences. In some aspects, a linker can be positioned between two nucleic acid sequences that modulate a TLR, such that from 5′ to 3′ the construct comprises a first nucleic acid sequence that modulates a TLR, a linker, and a second nucleic acid sequence that modulates a TLR. In some aspects, a nucleic acid sequence that modulates a TLR can be positioned” downstream or upstream from the 5′ or 3′ end of a first or a second ITR within a backbone.

As used here the term “tandem repeat” refers to a sequence of two or more nucleic acid sequences (e.g., a 2-40 nt length nucleic acid segment) that is repeated in such a way that each repeat is adjacent to one or two other repeats.

As used herein, the term “positioned in tandem” generally refers to two desired sequences, which are separated by some distance, e.g., separated by a linker as described herein.

As used herein, the term “rep genes” generally refers to an open reading frame which encodes replication gene products, e.g., AAV replication gene products. In some aspects, a family of at least four viral proteins are expressed from the rep genes of an AAV rep region: Rep 78, Rep 68, Rep 52, and Rep 40, all of which are named for their apparent molecular weights.

As used herein, the term “cap genes” generally refers to an open reading frame which encodes capsid (cap) regions, e.g., AAV capsid regions. In some aspects, the AAV cap region encodes at least three proteins: VP1, VP2, and VP3.

As used herein, the term “adenovirus genes” generally refers to any genes native to an adenovirus, e.g., genes that naturally occur within an adenovirus genome. Generally, adenovirus genomes are linear, non-segmented double-stranded (ds) DNA molecules that are typically 26-46 Kbp long, containing approximately 23-46 protein-coding genes in some instances. Adenovirus genes generally comprise genes encoding the major proteins required for viral DNA replication and major structural components. Adenovirus genomes typically comprise inverted repeat sequences of up to 150 bp in length located at the ends of the viral genome that function as DNA replication origins. These sequences enable circularization of single-stranded DNA, leading to base paired panhandle regions that can also function as DNA replication origins. Furthermore, adenovirus genes are generally organized into transcription units within the adenovirus genome. The adenovirus genome generally comprises five early transcription units including early region 1A (E1A), E1B, E2, E3, and E4. Intermediate transcription units, including IX, IVa2, L4 intermediate, and E2 late, are transcribed at the onset of DNA replication. A single late transcription unit (major late) generates five populations of late mRNAs, L1-L5. Most of the adenovirus transcription units are transcribed by RNA polymerase II and give rise to multiple mRNAs that are differentiated by alternative splicing or alternative poly(A) sites. In many cases, these transcription units encode more than one protein with related functions.

### II. Vector Constructs Comprising Nucleic Acid Sequences that Modulate Toll-Like Receptors

Some aspects of the present disclosure relate to a vector construct comprising: (a) a polynucleotide comprising a promoter operably linked to a nucleic acid of interest (e.g., an expression cassette); (b) a first terminal repeat and a second terminal repeat (e.g., flanking the expression cassette); and (c) a backbone polynucleotide comprising a nucleic acid sequence that modulates a Toll-like receptor (TLR).

### II.A.1 Toll-Like Receptors

Toll-like receptors (TLRs) are a class of proteins that play a key role in the innate immune system. TLRs include single-pass membrane-spanning receptors that are expressed on the membranes of leukocytes including dendritic cells, macrophages, natural killer cells, cells of the adaptive immunity T cells, and B cells, and non-immune cells (epithelial and endothelial cells, and fibroblasts). Certain TLRs can recognize structurally conserved molecules derived from microbes, and for instance, once a microbe has breached physical barriers such as the skin or intestinal tract mucosa, it is recognized by TLRs, which can activate immune cell responses, e.g., inflammatory responses, e.g., stimulate production of NK cells. Known TLRs include TLR1, TLR2, TLR3, TLR4, TLR5, TLR6, TLR7, TLR8, TLR9, TLR10, TLR11, TLR12, and TLR13. TLR11, TLR12, and TLR13 are not known to be in humans. In some instances, upon activation, TLRs can recruit adaptor proteins, e.g., proteins that mediate other protein-protein interactions, within the cytosol of an immune cell in order to propagate an antigen-induced signal transduction pathway. These recruited proteins can then subsequently activate other downstream proteins that can further amplify the signal and ultimately lead to the upregulation or suppression of genes that orchestrate inflammatory responses and other transcriptional events. Some of these events can lead to cytokine production, proliferation, and survival, while others can lead to greater adaptive immunity. In some aspects, modulation of TLRs is desired, e.g., decreasing an inflammatory response associated with a TLR, e.g., preventing a cytokine storm associated with TLR signaling, e.g., increasing TLR signaling to increase cytokine production, e.g., increasing TLR signaling to increase NK cell production.

### TLR3

Toll-like receptor 3 (TLR3), also known as CD283 (cluster of differentiation 283), is a protein that in humans is encoded by the TLR3 gene. TLR3 is abundantly expressed in placenta and pancreas and is also expressed in the dendritic subpopulation of leukocytes. TLR3 is capable of recognizing dsRNA associated with viral infection, and is also capable of inducing the activation of IRF3 and NF-κB. In some instances, TLR3 uses TRIF as an adaptor protein. IRF3 is capable of ultimately inducing the production of type I interferons. As such TLR3 can play a role in host defense against viruses, e.g., dsRNA viruses, e.g., viruses that produce dsRNA as a replicative intermediate during virus replication. In mouse models, TLR3 displays a protective role in atherosclerosis, and activation of TLR3 signaling is associated with ischemic preconditioning-induced protection against brain ischemia and attenuation of reactive astrogliosis. Furthermore, TLR3 activation has been shown to promote hair follicle regeneration in skin wound healing.

### TLR4

Toll-like receptor 4 (TLR4), also known as CD284 (cluster of differentiation 284), is a protein that in humans is encoded by the TLR4 gene and is a transmembrane protein. Generally, TLR4 activation can stimulate an intracellular signaling pathway NF-κB and inflammatory cytokine production, which is responsible for activating the innate immune system. TLR4 can recognize lipopolysaccharide (LPS), a component present in many Gram-negative bacteria (e.g. Neisseria spp.) and select Gram-positive bacteria. Additional ligands of TLR4 also include several viral proteins, polysaccharide, and a variety of endogenous proteins such as low-density lipoprotein, beta-defensins, and heat shock protein. TLR4 expression can be detected on many tumor cells and cell lines. TLR4 signaling in tumors in humans is generally reported to be pro-carcinogenic, mainly as a result of the contributions of proinflammatory cytokine signaling to a tumor-promoting microenvironment. Furthermore, TLR4 is capable of activating MAPK and NF-κB pathways, which further implicates TLR4 signaling in regulation of carcinogenesis, in particular, through increased proliferation of tumor cells, apoptosis inhibition, and metastasis.

### TLR7

Toll-like receptor 7 (TLR7) is a protein that in humans is encoded by the TLR7 gene. TLR7 is capable of recognizing single-stranded RNA in endosomes, which is a common feature of viral genomes which are internalized by macrophages and dendritic cells. TLR7 is known to play a role in the pathogenesis of autoimmune disorders such as lupus as well as in the regulation of antiviral immunity. A TLR7 agonist, Aldara, an imidazoquinoline, has been approved for topical use in treating warts caused by papillomavirus and for actinic keratoses. TLR7 is capable of inducing production of anti-cancer cytokines such as interleukin-12.

### TLR8

Toll-like receptor 8 (TLR8), also known as CD288 (cluster of differentiation 288), is a protein that in humans is encoded by the TLR8 gene. TLR8 is capable of recognizing G-rich oligonucleotides, and is further capable of recognizing single stranded RNA (ssRNA). TLR8 activation can ultimately result in transcription factor NF-κB and antiviral responses. TLR8 agonists (e.g. VTX-2337) are currently being evaluated as immune stimulants in therapy, e.g., combination therapy, for various forms of cancer.

### TLR9

Toll-like receptor 9, also known as CD289 (cluster of differentiation 289), is a protein that in humans is encoded by the TLR9 gene.

TLR9 is expressed in immune system cells including dendritic cells, macrophages, natural killer cells, and other antigen presenting cells. TLR9 is capable of binding foreign DNA such as that present in bacteria and viruses, which binding can trigger signaling cascades that lead to a pro-inflammatory cytokine responses.

Modulation of TLR9 expression has been demonstrated in diseases such as cancer as well as in infections and damaged tissues. Modulated TLR9 expression has also been implicated in autoimmune diseases. Previous studies in mice and humans indicate that this receptor mediates cellular response to unmethylated CpG dinucleotides in bacterial DNA to mount an innate immune response (see Entrez Gene: TLR9 toll-like receptor 9, NCBI Gene ID 54106).

TLR9 is capable of being activated by unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, keratinocytes, melanocytes, and plasmacytoid dendritic cells. TLR9 signals leads to activation of the cells initiating pro-inflammatory reactions that result in the production of cytokines such as type-I interferon, IL-6, TNF and IL-12. TLR9 can recognize nucleotides other than unmethylated CpG present in bacterial or viral genomes (Notley, C. A., et al., Scientific Reports 7, 42204 (2017)).

TLR9 is implicated as playing a role in a number of diseases and conditions, including cancer, breast cancer, renal cell carcinoma, ovarian cancer, non-small cell lung cancer, glioma, systemic lupus erythematosus (SLE), erythema nodosum leprosum (ENL), autoimmune thyroid diseases, inflammation, and/or inflammatory diseases.

II.A.2 Nucleic Acid Sequences that Modulate Toll-Like Receptors

Some aspects of the present disclosure relate to a vector construct comprising: (a) a polynucleotide comprising a promoter operably linked to a nucleic acid of interest (e.g., an expression cassette); (b) a first terminal repeat and a second terminal repeat (e.g., flanking the expression cassette); and (c) a backbone polynucleotide comprising a nucleic acid sequence that modulates a TLR. In some aspects, the nucleic acid sequence that modulates a Toll-like receptor can have immunologic effects, such as stimulating monocytes, macrophages, and dendritic cells that then produce several cytokines, including the TH1 cytokine interleukin 12.

In some aspects, the nucleic acid sequence that modulates a TLR comprises one or more of SEQ ID NOs: 1-28 or any of the sequences shown in Table 1, SEQ ID NOs: 29-38 or any of the sequences shown in Table 2, or SEQ ID NO: 54 or 55, or repeats thereof. In some aspects, the one or more nucleic acid sequences that modify a TLR share at least 85%, at least 90%, at least 95%, at least 96%, at least 97%, at least 98%, and/or at least 99% sequence identity with any one of SEQ ID NOs. 1-28 or any of the sequences shown in Table 1, or with any one of SEQ ID NOs: 29-38 or any of the sequences shown in Table 2. In some aspects, the nucleic acid sequence that modulates at TLR comprises one or more CpG ODNs. In some aspects, the first and/or second terminal repeat comprises a sequence selected from any one of SEQ ID Nos: 29-38 or any of the sequences show in Table 2.

In some aspects, the ODNs incorporated into a vector construct backbone as disclosed herein can modulate a TLR response in a cell transduced with the vector construct or portion of thereof and/or a virus particle comprising a portion of the vector construct. For example, the ODN can comprise between between 2 and 500, between 2 and 400, between 2 and 300, between 2 and 200, between 2 and 100, between 2 and 50, between 2 and 48, between 2 and 24, or between 2 and 6 nucleotides. In some aspects, the ODN comprise sequences that have a stimulatory effect on a TLR. In some aspects, the ODN comprise sequences that have an inhibitory effect on a TLR. In some aspects, the ODN comprises one or more tandem repeats. In some aspects, the ODN comprise at least two tandem repeats of a sequence. In some aspects, the ODN comprise at least two tandem repeats of a “TTAGGG” (SEQ ID NO: 55) sequence. In some aspects, the ODN comprises SEQ ID NO: 3. In some aspects, the backbone polynucleotide comprise two or more copies of the ODN. In some aspects, one or more of the copies of the ODN are packaged in a viral particular (e.g., AAV). In some aspects, the ODN comprise repeats of TTAGGG (SEQ ID NO: 55), e.g., 2 to 40 repeats, 2 to 35 repeats, 2 to 30 repeats, 2 to 25 repeats, 2 to 20 repeats, 2 to 18 repeats, 2 to 16 repeats, 4 to 20 repeats, 4 to 18 repeats, or 4 to 16 repeats. In some aspects, the vector backbone comprises two or more copies of one or more ODNs disclosed herein. In some aspects, the ODN comprise repeats of TTAGGG (SEQ ID NO: 55) in opposite orientations outside of the 5′ and the 3′ ends of the ITRs. In some aspects, the ODNs can be in the same or opposite orientations.

In some aspects, the backbone polynucleotide of the vector construct comprises 2 or more nucleic acids that modulate a TLR. In some aspects, the nucleic acids that modulate the TLR are the same sequence (e.g., multiple copies of the same sequence that modulates the same TLR). In some aspect, the nucleic acids that modulate the TLR are different sequences (e.g., different sequences that modulate the same TLR, or different sequences that modulate one or more different TLRs). In some aspects, the backbone polynucleotide comprises 2 or more, 3 or more, 4, or more, 5 or more, 10 or more, 20 or more, 30 or more, 40 or more, 50 or more, 50 or more, 70 or more, 80 or more, 90 or more, 100 or more, 150 or more, 200 or more, 250 or more, 300 or more, 350 or more, 400 or more, 450 or more, or 500 nucleic acids that modulate a TLR. In some aspects, the backbone polynucleotide comprises between 2 to 500 copies, between 2 to 450 copies, between 2 to 400 copies, between 2 to 350 copies, between 2 to 300 copies, between 2 to 250 copies, between 2 to 200 copies, between 2 to 150 copies, between 2 to 100 copies, between 2 to 95 copies, between 2 to 90 copies, between 2 to 85 copies, between 2 to 80 copies, between 2 to 75 copies, between 2 to 70 copies, between 2 to 65 copies, between 2 to 60 copies, between 2 to 55 copies, between 2 to 50 copies, between 2 to 45 copies, between 2 to 40 copies, between 2 to 35 copies, between 2 to 30 copies, between 2 to 25 copies, between 2 to 20 copies, between 2 to 15 copies, between 2 to 10 copies, between 2 to 9 copies, between 2 to 8 copies, between 2 to 7 copies, between 2 to 6 copies, between 2 to 5 copies, between 2 to 4 copies, or between 2 to 3 copies of a polynucleotide that modulates a TLR. In some aspects, the backbone polynucleotide comprises between 2 to 500 copies, between 2 to 200 copies, between 2 to 150 copies, between 2 to 100 copies, between 2 to 50 copies, between 2 to 40 copies, between 2 to 30 copies, between 2 to 25 copies, between 2 to 20 copies, between, 2 to 15 copies, or between 2 to 10 nucleic acids that modulates a TLR. In some aspects, the nucleic acid that modulates a TLR comprises repeats of SEQ ID NO: 55 (e.g., SEQ ID NO: 3).

In some aspects, a polynucleotide that modulates a TLR is positioned at any location within a backbone of a vector construct as described herein. In some aspects, a nucleic acid that modulates a TLR is positioned within 500 nucleotides or less, 450 nucleotides or less, 400 nucleotides or less, 350 nucleotides or less, 300 nucleotides or less, 250 nucleotides or less, 200 nucleotides or less, 150 nucleotides or less, 100 nucleotides or less, or 50 nucleotides or less from the first terminal repeat (e.g., ITR) or the second terminal repeat (e.g., ITR) of a vector construct disclosed herein. In some aspects, a nucleic acid that modulates a TLR is positioned within 2000 nucleotides or less, 1900 nucleotides or less, 1800 nucleotides or less, 1700 nucleotides or less, 1600 nucleotides or less, 1500 nucleotides or less, 1400 nucleotides or less, 1300 nucleotides or less, 1200 nucleotides or less, 1100 nucleotides or less, 1000 nucleotides or less, 950 nucleotides or less, 900 nucleotides or less, 850 nucleotides or less, 800 nucleotides or less, 750 nucleotides or less, 700 nucleotides or less, 650 nucleotides or less, 600 nucleotides or less, 550 nucleotides or less, 500 nucleotides or less, 450 nucleotides or less, 400 nucleotides or less, 350 nucleotides or less, 300 nucleotides or less, 250 nucleotides or less, 200 nucleotides or less, 190 nucleotides or less, 180 nucleotides or less, 170 nucleotides or less, 160 nucleotides or less, 150 nucleotides or less, 140 nucleotides or less, 130 nucleotides or less, 120 nucleotides or less, 110 nucleotides or less, 100 nucleotides or less, 95 nucleotides or less, 90 nucleotides or less, 85 nucleotides or less, 80 nucleotides or less, 75 nucleotides or less, 70 nucleotides or less, 65 nucleotides or less, 60 nucleotides or less, 55 nucleotides or less, 50 nucleotides or less, 45 nucleotides or less, 40 nucleotides or less, 35 nucleotides or less, 30 nucleotides or less, 25 nucleotides or less, 20 nucleotides or less, 15 nucleotides or less, 10 nucleotides or less, 9 nucleotides or less, 8 nucleotides or less, 7 nucleotides or less, 6 nucleotides or less, or 5 nucleotides or less from the first terminal repeat (e.g., ITR) or the second terminal repeat (e.g., ITR) of a vector construct disclosed herein. In some aspects, a nucleic acid that modulates a TLR is positioned adjacent to the first terminal repeat (e.g., ITR) and/or the second terminal repeat (e.g., ITR).

In some aspects, the nucleic acid sequence that modulates the TLR comprises about 0.1% to about 90%, about 0.1% to about 80%, about 0.1% to about 70%, about 0.1% to about 60%, about 0.1% to about 50%, about 0.1% to about 40%, about 0.1% to about 30%, about 0.1% to about 20%, about 0.1% to about 10%, about 0.1% to about 5%, about 0.1% to about 4%, about 0.1% to about 3%, about 0.1% to about 1%, or about 0.5% to about 1%, about 0.5% to about 10%, about 0.5% to about 5%, about 0.5% to about 4%, about 0.5% to about 3%, about 0.5% to about 2%, or about 0.5% to about 1% of the total nucleic acid sequence of the backbone polynucleotide of a vector construct. In some aspects, the nucleic acid sequence that modulates the TLR comprises about 0.1% or more, about 0.2% or more, about 0.3% or more, about 0.4% or more, about 0.5% or more, about 0.6% or more, about 0.7% or more, about 0.8% or more, about 0.9% or more, about 1.0% or more, about 2% or more, about 3% or more, about 4% or more, about 5% or more, about 6% or more, about 7% or more, about 8% or more, about 9% or more, about 10% or more, about 15% or more, about 20% or more, about 30% or more, about 40% or more, about 50% or more, about 60% or more, about 70% or more, about 80% or more, or about 90% or more of the total nucleic acid sequence of the backbone polynucleotide of a vector construct.

In some aspects, the polynucleotide that modulates the TLR comprises any one or more of the sequences of Table 1.

TABLE 1

| | | Nucleic Acid |
|---|---|---|
| SEQ ID NO | Nucleic Acid Sequence | Sequence Target |
| 1 | TCCTGGAGGGGAAGT | TLR9 |
| 2 | CCTTGGATGGGAA | TLR9 |
| 3 | TTAGGGTTAGGGTTAGGGTTAGGG | TLR9 |
| 4 | TCCTGGATGGGAAGT | TLR9 |
| 5 | TTCCCATCCAGGCCTGGATGGGAA | TLR9 |
| 6 | CTTACCGCTGCACCTGGATGGGAA | TLR9 |
| 7 | GGGGGGGGGGGGGGGGGGGG | TLR9 |
| 8 | TGACTGTGAAGGTTAGAGATGA | TLR9 |
| 9 | TCCTGGAGGGGTTGT | TLR9 |
| 10 | CCTGGCGGGG | TLR9 |
| 11 | CCTGGATGGGAATTCCCATCCAGG | TLR9 |
| 12 | CCTGGATGGGAACTTACCGCTGCA | TLR9 |
| 13 | CTCCTATTGGGGGTTTCCTAT | TLR9 |
| 14 | TCGTATCCTGGAGGGGAAG | TLR9 |
| 15 | CCTATCCTGGAGGGGAAG | TLR9 |
| 16 | TAATATCCTGGAGGGGAAG | TLR9 |
| 17 | TCCTATCCTGGAGGGGAAG | TLR9 |
| 18 | TCCTGGCGGGGAAGT | TLR7, TLR8, and TLR9 |
| 19 | TCCTGGCG | TLR 7 and TLR9 |
| 20 | TCGTCGTTCTG | TLR 7 and TLR9 |
| 21* | CTGTCGTTCTC-XN-CTCTTGCTGTC | TLR 7, TLR8, andTLR9 |
| 22* | TCGTCGTTCTG-XN-GTCTTGCTGCT | TLR7 and TLR9 |
| 23 | TGGCGCGCACCCACGGCCTG | TLR3, TLR7, andTLR9 |
| 24 | CTTCGCGTTAGCGTTGAGTG | TLR9 |
| 25 | TTGCGCGCCCTCCATCGAGG | TLR9 |
| 26 | TGCTCCTGGAGGGGTTGT | TLR7 and TLR9 |
| 27 | TGCTTGCAAGCTTGCAAGCAGCGGATGAAGACTGGTG | TLR7 |
| | TGCCAATAAACCATATC | |
| 28 | GCCGCGTTAGCATGTACTCGGTTGGCCCTAAATACGA | TLR4 |
| | GCAAC | |

*Regarding SEQ ID NO: 21 and SEQ ID NO: 22: X can be any nucleotide, e.g., X can
be A, T, G, or C; and N = 0 or more nucleotides in length.

In some aspects, the nucleic acid that modulates a TLR comprises any one or more of SEQ ID NOs: 1-28 or any of the sequences shown in Table 1. In some aspects, the nucleic acid that modulates a TLR comprises two or more repeats of SEQ ID NO: 54 or SEQ ID NO: 55. In some aspects, the nucleic acid that modulates a TLR comprises two or more repeats of SEQ ID NO: 55 (e.g., SEQ ID NO: 3). In some aspects, the nucleic acid that modulates a TLR comprises at least one, two, three, four, five, six, seven, eight, nine, or ten copies of SEQ ID NO: 3. In some aspects, the nucleic acid that modulates a TLR comprises at least one, two, three, four, five, six, seven, eight, nine, or ten of one or more copies of SEQ ID NOs: 1, 3, 12, 18, and 26, or any combination thereof. In some aspects, the nucleic acid that modulates a TLR comprises at least one, two, three, four, five, six, seven, eight, nine, or ten of one or more of SEQ ID NOs: 1, 2, 3, 12, 16, and 17, or any combination thereof. In some aspects, the nucleic acid that modulates a TLR comprises at least one, two, three, four, five, six, seven, eight, nine, or ten copies of one or more of SEQ ID NOs: 18, 20, 23, and 26, or any combination thereof. In some aspects, the nucleic acid that modulates a TLR comprises at least one, two, three, four, five, six, seven, eight, nine, or ten copies of one or more of SEQ ID NOs: 1, 2, 3, 12, 16, 17, 20, and 26, or any combination thereof.

In some aspects, the nucleic acid that modulates a TLR comprises SEQ ID NO: 21 and/or SEQ ID NO: 22. In some aspects, the “X” in each of SEQ ID NO: 21 and SEQ ID NO: 22 is any nucleotide, e.g., X can be A, T, G, or C. In some aspects, the “X” in each each of SEQ ID NO: 21 and SEQ ID NO: 22 is any nucleotide, e.g., X can be A, T, G, or C, and furthermore X is 0 or more nucleotides in length. In some aspects, the “X” in each of SEQ ID NO: 21 and SEQ ID NO: 22 is any nucleotide, e.g., X can be A, T, G, or C, and furthermore the N of SEQ ID NO: 21 and/or SEQ ID NO: 22 is between 0 and 5000, between 0 and 4750, between 0 and 4500, between 0 and 4250, between 0 and 4000, between 0 and 3750, between 0 and 3500, between 0 and 3250, between 0 and 3000, between 0 and 2750, between 0 and 2500, between 0 and 2250, between 0 and 2000, between 0 and 1750, between 0 and 1500, between 0 and 1250, between 0 and 1000, between 0 and 975, between 0 and 950, between 0 and 925, between 0 and 900, between 0 and 875, between 0 and 850, between 0 and 825, between 0 and 800, between 0 and 775, between 0 and 750, between 0 and 725, between 0 and 700, between 0 and 675, between 0 and 650, between 0 and 625, between 0 and 600, between 0 and 575, between 0 and 550, between 0 and 525, between 0 and 500, between 0 and 475, between 0 and 450, between 0 and 425, between 0 and 400, between 0 and 375, between 0 and 350, between 0 and 325, between 0 and 300, between 0 and 275, between 0 and 250, between 0 and 225, between 0 and 200, between 0 and 175, between 0 and 150, between 0 and 125, between 0 and 100, between 0 and 95, between 0 and 90, between 0 and 85, between 0 and 80, between 0 and 75, between 0 and 70, between 0 and 65, between 0 and 60, between 0 and 55, between 0 and 50, between 0 and 45, between 0 and 40, between 0 and 35, between 0 and 30, between 0 and 29, between 0 and 28, between 0 and 27, between 0 and 26, between 0 and 25, between 0 and 24, between 0 and 23, between 0 and 22, between 0 and 21, between 5 and 5000, between 5 and 4750, between 5 and 4500, between 5 and 4250, between 5 and 4000, between 5 and 3750, between 5 and 3500, between 5 and 3250, between 5 and 3000, between 5 and 2750, between 5 and 2500, between 5 and 2250, between 5 and 2000, between 5 and 1750, between 5 and 1500, between 5 and 1250, between 5 and 1000, between 5 and 975, between 5 and 950, between 5 and 925, between 5 and 900, between 5 and 875, between 5 and 850, between 5 and 825, between 5 and 800, between 5 and 775, between 5 and 750, between 5 and 725, between 5 and 700, between 5 and 675, between 5 and 650, between 5 and 625, between 5 and 600, between 5 and 575, between 5 and 550, between 5 and 525, between 5 and 500, between 5 and 475, between 5 and 450, between 5 and 425, between 5 and 400, between 5 and 375, between 5 and 350, between 5 and 325, between 5 and 300, between 5 and 275, between 5 and 250, between 5 and 225, between 5 and 200, between 5 and 175, between 5 and 150, between 5 and 125, between 5 and 100, between 5 and 95, between 5 and 90, between 5 and 85, between 5 and 80, between 5 and 75, between 5 and 70, between 5 and 65, between 5 and 60, between 5 and 55, between 5 and 50, between 5 and 45, between 5 and 40, between 5 and 35, between 5 and 30, between 5 and 29, between 5 and 28, between 5 and 27, between 5 and 26, between 5 and 25, between 5 and 24, between 5 and 23, between 5 and 22, between 5 and 21, between 10 and 5000, between 10 and 4750, between 10 and 4500, between 10 and 4250, between 10 and 4000, between 10 and 3750, between 10 and 3500, between 10 and 3250, between 10 and 3000, between 10 and 2750, between 10 and 2500, between 10 and 2250, between 10 and 2000, between 10 and 1750, between 10 and 1500, between 10 and 1250, between 10 and 1000, between 10 and 975, between 10 and 950, between 10 and 925, between 10 and 900, between 10 and 875, between 10 and 850, between 10 and 825, between 10 and 800, between 10 and 775, between 10 and 750, between 10 and 725, between 10 and 700, between 10 and 675, between 10 and 650, between 10 and 625, between 10 and 600, between 10 and 575, between 10 and 550, between 10 and 525, between 10 and 500, between 10 and 475, between 10 and 450, between 10 and 425, between 10 and 400, between 10 and 375, between 10 and 350, between 10 and 325, between 10 and 300, between 10 and 275, between 10 and 250, between 10 and 225, between 10 and 200, between 10 and 175, between 10 and 150, between 10 and 125, between 10 and 100, between 10 and 95, between 10 and 90, between 10 and 85, between 10 and 80, between 10 and 75, between 10 and 70, between 10 and 65, between 10 and 60, between 10 and 55, between 10 and 50, between 10 and 45, between 10 and 40, between 10 and 35, between 10 and 30, between 10 and 29, between 10 and 28, between 10 and 27, between 10 and 26, between 10 and 25, between 10 and 24, between 10 and 23, between 10 and 22, or between 10 and 21 nucleotides in length.

In some aspects, the vector construct comprises any one or more of the sequences of Table 2.

TABLE 2

| | TERMINAL REPEAT | | |
|---|---|---|---|
| SEQ | SEQUENCE NEAR | SEQ | TERMINAL REPEAT |
| ID NO | PROMOTER | ID NO | SEQUENCE NEAR POLYA |
| 29 | CCCTAACCCTAACCCTA | 30 | TTAGGGTTAGGGTTAGGGT |
| | ACCCTAACCCTAACCCT | | TAGGGTTAGGGTTAGGGTT |
| | AACCCTAACCCTAACCC | | AGGGTTAGGGTTAGGGTTA |
| | TAACCCTAACCCTAACC | | GGGTTAGGGTTAGGG |
| | CTAA | | |
| 31 | ACTTCCCCTCCAGGATG | 32 | TTAGGGTTAGGGTTAGGGT |
| | CAGCGGTAAGTTCCCAT | | TAGGGTCCTGGCGGGGAAG |
| | CCAGGACAACCCCTCCA | | TTGCTCCTGGAGGGGTTGT |
| | GGAGCAACTTCCCCGCC | | CCTGGATGGGAACTTACCG |
| | AGGACCCTAACCCTAAC | | CTGCATCCTGGAGGGGAAG |
| | CCTAACCCTAA | | T |
| 33 | TTCCCATCCAAGGCTTC | 34 | TTAGGGTTAGGGTTAGGGT |
| | CCCTCCAGGATAGGACT | | TAGGGCCTGGATGGGAACT |
| | TCCCCTCCAGGATATTA | | TACCGCTGCATCCTGGAGG |
| | ACTTCCCCTCCAGGATG | | GGAAGTTAATATCCTGGAG |
| | CAGCGGTAAGTTCCCAT | | GGGAAGTCCTATCCTGGAG |
| | CCAGGCCCTAACCCTAA | | GGGAAGCCTTGGATGGGAA |
| | CCCTAACCCTAA | | |
| 35 | CAGGCCGTGGGTGCGCG | 36 | TCCTGGCGGGGAAGTTGCT |
| | CCACAGAACGACGAACA | | CCTGGAGGGGTTGTTCGTC |
| | ACCCCTCCAGGAGCAAC | | GTTCTGTGGCGCGCACCCA |
| | TTCCCCGCCAGGA | | CGGCCTG |
| 37 | CAGAACGACGATTCCCA | 38 | TTAGGGTTAGGGTTAGGGT |
| | TCCAAGGCTTCCCCTCC | | TAGGGTGCTCCTGGAGGGG |
| | AGGATAGGACTTCCCCT | | TTGTCCTGGATGGGAACTT |
| | CCAGGATATTAACTTCC | | ACCGCTGCATCCTGGAGGG |
| | CCTCCAGGATGCAGCGG | | GAAGTTAATATCCTGGAGG |
| | TAAGTTCCCATCCAGGA | | GGAAGTCCTATCCTGGAGG |
| | CAACCCCTCCAGGAGCA | | GGAAGCCTTGGATGGGAAT |
| | CCCTAACCCTAACCCTA | | CGTCGTTCTG |
| | ACCCTAA | | |

In some aspects, the polynucleotide that modulates the TLR are of any origin, e.g., bacterial, human, synthetic, and/or from other sources. In some aspects, the polynucleotide that modulates the TLR shares at least 85%, at least 90%, at least 95%, at least 96%, at least 97%, at least 98%, and/or at least 99% with any one of SEQ ID NOs. 1-28 or any of the sequences shown in Table 1. In some aspects, the first and/or second terminal repeat comprises a sequence that shares at least 85%, at least 90%, at least 95%, at least 96%, at least 97%, at least 98%, and/or at least 99% with any one of SEQ ID Nos: 29-38 or any of the sequences show in Table 2. In some aspects, the polynucleotide that modulates the TLR are of any origin, e.g., bacterial, human, synthetic, and/or from other sources. In some aspects, the polynucleotide that modulates the TLR shares at least 85%, at least 90%, at least 95%, at least 96%, at least 97%, at least 98%, and/or at least 99% with any one of SEQ ID NOs. 29-38. In some aspects, any one or more of SEQ ID NOs: 1-38 are used as a part of a terminal repeat, e.g., a long terminal repeat, e.g., a long terminal repeat, and/or near a terminal repeat, e.g., as discussed herein.

In some aspects, a nucleic acid that modulates the TLR is introduced into host cells as part of the viral genome or virion, rather than as a separate agent. In some aspects, this renders the effect of the oligonucleotide sequences local rather than systemic. In some aspects, the immune evasion is transient as it occurs during AAV or other virus entry, unlike immune suppression with drugs which can last for weeks. In some aspects, if the virus or viral genome is not replicated in the host cell, then the effect of the nucleic acid will be transient. In some aspects, if the virus or viral genome is replicated, the effect will be coextensive with the replication.

In some aspects, a nucleic acid that modulates the TLR is inserted into a viral genome using recombinant DNA engineering. In some instances, a nucleic acid that modulates the TLR is located in an untranslated region of the viral genome.

In some aspects, constructs comprising a nucleic acid that modulates the TLR demonstrate any one or more of the following features: (a) does not lower viral packaging and infectivity, (b) prevents TLR9-mediated inflammation, (c) reduces induction of pro-inflammatory cytokines, and (d) increases expression of nucleic acid of interest. The increased nucleic acid of interest expression can be due to a reduced immune response.

In some aspects, a nucleic acid that modulates the TLR is incorporated into a virus that has potential utility for humans and other mammals but elicit inflammatory/immune responses that are undesirable. For example, oncolytic viruses that preferentially infect and lyse cancer cells are used to kill or shrink tumors. These viruses are replicative (unlike AAV vectors used for gene therapy) so they can release new virions to shrink the remaining tumor. Examples include wild-type or variants of herpes simplex virus, adenovirus, and enterovirus.

In some aspects, a nucleic acid that modulates a TLR is packaged into one or more viral capsids. In some instances, the packaging can occur during recombinant virus production. In some aspects, the nucleic acid that modulates a TLR can be part of a partial backbone sequence linked to a terminal repeat sequence (e.g., ITR) (alone or further comprising the open reading frame sequence) packaged into recombinant virions. In some aspects, the nucleic acid that modulates a TLR can be part of backbone DNA fragments packaged into recombinant virions (without ITR or open reading frame sequences). See FIGS. 3A and 3B for an exemplary illustrations.

In some aspects, the viral vector construct comprises a linker positioned between the two or more of the nucleic acid sequences that modulates the TLR. In some aspects, the linker comprises one or more nucleotides. In some aspects, the linker comprises one or more nucleotide analogues and/or synthetic nucleotides known in the art. In some aspects, the linker comprises DNA and/or RNA bases. In some aspects, the linker is 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more nucleotides in length. In some aspects, the linker comprises DNA and/or RNA bases. In some aspects, the linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some aspects, the linker is 1 to 30, 1 to 29, 1 to 28, 1 to 27, 1 to 26, 1 to 25, 1 to 24, 1 to 23, 1 to 22, 1 to 21, 1 to 20, 2 to 30, 2 to 29, 2 to 28, 2 to 27, 2 to 26, 2 to 25, 2 to 24, 2 to 23, 2 to 22, 2 to 21, 2 to 20, 3 to 30, 3 to 29, 3 to 28, 3 to 27, 3 to 26, 3 to 25, 3 to 24, 3 to 23, 3 to 22, 3 to 21, or 3 to 20 nucleotides in length. In some aspects, the linker is 3 to 25 nucleotides in length. In some aspects, the linker comprises a sequence selected from the sequences of Table 3. In some aspects, the linker comprises a sequence selected from the group consisting of SEQ ID NOs: 39-46.

TABLE 3

| SEQ ID NO | Spacer Sequence |
|---|---|
| 39 | AAAAA |
| 40 | ATTGATATTCGTTGGTTTTG |
| 41 | AATTGATATTCGTTGGTTTT |
| 42 | TTGGAAGAAATTGATATTCGT |
| 43 | GAGTGAATTATGAATGAAAT |
| 44 | AAAGCTTTTGAGGTTGGTGG |
| 45 | TCATCTTTACCGGTCATGGA |
| 46 | TCTTTACCGGTCATGGGACGG |

### II.A.3 Backbone Polynucleotides

Some aspects of the present disclosure relate to vector construct comprising: (a) a polynucleotide comprising a promoter operably linked to a nucleic acid of interest; (b) a first terminal repeat and a second terminal repeat; and (c) a backbone polynucleotide comprising a nucleic acid sequence that modulates a TLR. In some aspects, the portion or fragment of the backbone comprising a nucleic acid sequence that modulates a TLR can be linked to a terminal repeat (e.g., ITR) sequence or can be a portion or fragment of the backbone that does not comprise a terminal repeat (e.g., ITR) sequence.

In some aspects, the backbone polynucleotide is engineered to encode one or more selectable markers or reporters that provide for the selection or identification of cells that have incorporated the polynucleotide-based vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), 0-galactosidase (LacZ), 0-glucuronidase (Gus), and the like.

In some aspects, the backbone polynucleotide is at least 4000 nucleotides in length. In some aspects, the backbone polynucleotide is about 1000, about 1250, about 1500, about 1750, about 2000, about 2250, about 2500, about 2750, about 3000, about 3250, about 3500, about 3750, about 4000, about 4250, about 4500, about 4750, about 5000, about 5250, about 5500, about 5750, about 6000, about 6250, about 6500, about 6750, about 7000, about 7250, about 7500, about 7750, or about 8000 nucleotides in length. In some aspects, the backbone polynucleotide is about 2000 to about 8000, about 2000 to 7000, about 2000 to about 6000, about 2000 to about 5000, about 2000 to about 4000, about 3000 to about 8000, about 3000 to about 7000, about 3000 to about 6000, about 3000 to about 5000, about 3000 to about 4000, about 4000 to about 8000, about 4000 to about 7000, about 4000 to about 6000, about 4000 to about 5000, about 5000 to about 8000, about 5000 to about 7000, about 5000 to about 6000 nucleotides in length.

In some aspects, the backbone polynucleotide comprises 2 or more copies of a polynucleotide that modulates a TLR. In some aspects, the backbone polynucleotide comprises 2 or more, 3 or more, 4, or more, 5 or more, 10 or more, 20 or more, 30 or more, 40 or more, 50 or more, 50 or more, 70 or more, 80 or more, 90 or more, 100 or more, 150 or more, 200 or more, 250 or more, 300 or more, 350 or more, 400 or more, 450 or more, or 500 copies of a polynucleotide that modulates a TLR. In some aspects, the backbone polynucleotide comprises between 2 to 500 copies, between 2 to 450 copies, between 2 to 400 copies, between 2 to 350 copies, between 2 to 300 copies, between 2 to 250 copies, between 2 to 200 copies, between 2 to 150 copies, between 2 to 100 copies, between 2 to 95 copies, between 2 to 90 copies, between 2 to 85 copies, between 2 to 80 copies, between 2 to 75 copies, between 2 to 70 copies, between 2 to 65 copies, between 2 to 60 copies, between 2 to 55 copies, between 2 to 50 copies, between 2 to 45 copies, between 2 to 40 copies, between 2 to 35 copies, between 2 to 30 copies, between 2 to 25 copies, between 2 to 20 copies, between 2 to 15 copies, between 2 to 10 copies, between 2 to 9 copies, between 2 to 8 copies, between 2 to 7 copies, between 2 to 6 copies, between 2 to 5 copies, between 2 to 4 copies, or between 2 to 3 copies of a polynucleotide that modulates a TLR. IN some aspects, the backbone polynucleotide comprises between 2 to 500 copies, between 2 to 200 copies, between 2 to 150 copies, between 2 to 100 copies, between 2 to 50 copies, between 2 to 40 copies, between 2 to 30 copies, between 2 to 25 copies, between 2 to 20 copies, between, 2 to 15 copies, or between 2 to 10 copies of a polynucleotide that modulates a TLR.

### III. Constructs

Some aspects of the disclosure are directed to a nucleic acid construct or an expression construct (e.g., comprising an expression cassette) comprising a eukaryotic promoter operably linked to a nucleic acid of interest. In some aspects, the constructs containing the DNA sequence (or the corresponding RNA sequence) which can be used in accordance with the disclosure can be any eukaryotic expression construct containing the nucleic acid, e.g., DNA or the RNA sequence, of interest. For example, a plasmid or vector construct can be cleaved to provide linear DNA having ligatable termini. These termini are bound to exogenous DNA having complementary, like ligatable termini to provide a biologically functional recombinant DNA molecule having an intact replicon and a desired phenotypic property. In some aspects, the construct is capable of replication in both eukaryotic and prokaryotic hosts, which constructs are known in the art and are commercially available

The exogenous (i.e., donor) DNA used in the disclosure is obtained from suitable cells, and the constructs prepared using techniques well known in the art. Likewise, techniques for obtaining expression of exogenous DNA or RNA sequences in a genetically altered host cell are known in the art (see e.g., Kormal et al., Proc. Natl. Acad. Sci. USA, 84:2150-2154 (1987); Sambrook et al. Molecular Cloning: a Laboratory Manual, 2nd Ed., 1989, Cold Spring Harbor Laboratory Press, Cold Spring Harbor, N.Y.; each of which are hereby incorporated by reference with respect to methods and compositions for eukaryotic expression of a DNA of interest).

In some aspects, the nucleic acid construct or expression construct (e.g., comprising an expression cassette) comprises a promoter operably linked to a nucleic acid of interest which promoter and nucleic acid of interest are located between a first and a second terminal repeat and comprises a length less than or equal to a viral genome. In some aspects, the nucleic acid construct or expression construct comprises a eukaryotic promoter operably linked to a nucleic acid of interest, which promoter and nucleic acid of interest are located between a first and a second terminal repeat and comprises a length less than a viral genome.

In some aspects, the nucleic acid construct or expression construct is located between a first and a second terminal repeat.

In some aspects, the nucleic acid construct or expression construct comprises a length less than or equal to a single-stranded AAV genome.

In some aspects, the nucleic acid construct or expression construct comprises a length less than or equal to a self-complementary AAV genome.

In some aspects, the nucleic acid construct or expression construct is less than about 4700 nucleotides in length.

In some aspects, the nucleic acid construct or expression construct is less than about 2300 nucleotides in length.

In some aspects, the length of the expression cassette will be less than the length of a wildtype single-stranded AAV genome (about 4.7 kb) in order to increase the frequency of packaging of the backbone (e.g., about 2.0 kb). In some aspects, the length of the expression cassette will be less than the length of a wildtype single-stranded AAV genome (e.g., less than about 4.7 kb). In some aspects, the length of the backbone packaged will comprise between about 0.2 kb to 3.0 kb, about 0.2 kb to 2.5 kb, about 1.0 kb to 2.2 kb, about 1.0 kb to 2.0 kb, about 1.0 kb to 1.8 kb, about 1.0 kb to 1.4 kb, about 2.2 kb, about 2.0 kb, about 1.8 kb, about 1.6 kb, about 1.4 kb, about 1.2 kb, about 1 kb, about 0.8 kb or about 0.5 kb, or any combination thereof.

In some aspects, the length of the expression cassette will be less than the length of a wildtype self-complementary AAV genome (about 2.3 kb) in order to increase the frequency of packaging of the backbone (e.g., about 1.0 kb). In some aspects, the length of the expression cassette will be less than the length of a wildtype self-complementary AAV genome (e.g., less than about 2.3 kb). In some aspects, the backbone packaged will be between about 1.5 kb and 0.1 kb, about 1.2 kb and 0.1 kb, about 1.2 kb and 0.5 kb, about 1 kb, about 0.8 kb, about 0.6 kb, about 0.4 kb, about 0.2 kb, about 0.1 kb, or any combination thereof.

In some aspects, the nucleic acid construct or expression construct is less than 4650 nucleotides, less than 4600 nucleotides, less than 4550 nucleotides, less than 4500 nucleotides, less than 4450 nucleotides, less than 4400 nucleotides, less than 4350 nucleotides, less than 4300 nucleotides, less than 4250 nucleotides, less than 4200 nucleotides, less than 4150 nucleotides, less than 4100 nucleotides, less than 4050 nucleotides, less than 4000 nucleotides, less than 3950 nucleotides, less than 3900 nucleotides, less than 3850 nucleotides, less than 3800 nucleotides, less than 3750 nucleotides, less than 3700 nucleotides, less than 3650 nucleotides, less than 3600 nucleotides, less than 3550 nucleotides, less than 3500 nucleotides, less than 3450 nucleotides, less than 3400 nucleotides, less than 3350 nucleotides, less than 3300 nucleotides, less than 3250 nucleotides, less than 3200 nucleotides, less than 3150 nucleotides, less than 3100 nucleotides, less than 3050 nucleotides, less than 3000 nucleotides, less than 2950 nucleotides, less than 2900 nucleotides, less than 2850 nucleotides, less than 2800 nucleotides, less than 2750 nucleotides, less than 2700 nucleotides, less than 2650 nucleotides, less than 2600 nucleotides, less than 2550 nucleotides, less than 2500 nucleotides, less than 2450 nucleotides, less than 2400 nucleotides, less than 2350 nucleotides, less than 2300 nucleotides, less than 2250 nucleotides, less than 2200 nucleotides, less than 2150 nucleotides, less than 2100 nucleotides, less than 2050 nucleotides, less than 2000 nucleotides, less than 1950 nucleotides, less than 1900 nucleotides, less than 1850 nucleotides, less than 1800 nucleotides, less than 1750 nucleotides, less than 1700 nucleotides, less than 1650 nucleotides, less than 1600 nucleotides, less than 1550 nucleotides, less than 1500 nucleotides, less than 1450 nucleotides, less than 1400 nucleotides, less than 1350 nucleotides, less than 1300 nucleotides, less than 1250 nucleotides, less than 1200 nucleotides, less than 1150 nucleotides, less than 1100 nucleotides, less than 1050 nucleotides, or less than 1000 nucleotides.

In some aspects, the nucleic acid construct or expression construct comprises a eukaryotic promoter operably linked to a nucleic acid of interest, which promoter and nucleic acid of interest are located between a first and a second terminal repeat and measure in length less than an AAV genome and the nucleic acid that modulates a TLR is positioned within 500 nucleotides or less, 450 nucleotides or less, 400 nucleotides or less, 350 nucleotides or less, 300 nucleotides or less, 250 nucleotides or less, 200 nucleotides or less, 150 nucleotides or less, 100 nucleotides or less, or 50 nucleotides or less from the first terminal repeat (e.g., ITR) or the second terminal repeat (e.g., ITR) of a vector construct disclosed herein.

In some aspects, the nucleic acid that modulates a TLR is positioned within 2000 nucleotides or less, 1900 nucleotides or less, 1800 nucleotides or less, 1700 nucleotides or less, 1600 nucleotides or less, 1500 nucleotides or less, 1400 nucleotides or less, 1300 nucleotides or less, 1200 nucleotides or less, 1100 nucleotides or less, 1000 nucleotides or less, 950 nucleotides or less, 900 nucleotides or less, 850 nucleotides or less, 800 nucleotides or less, 750 nucleotides or less, 700 nucleotides or less, 650 nucleotides or less, 600 nucleotides or less, 550 nucleotides or less, 500 nucleotides or less, 450 nucleotides or less, 400 nucleotides or less, 350 nucleotides or less, 300 nucleotides or less, 250 nucleotides or less, 200 nucleotides or less, 190 nucleotides or less, 180 nucleotides or less, 170 nucleotides or less, 160 nucleotides or less, 150 nucleotides or less, 140 nucleotides or less, 130 nucleotides or less, 120 nucleotides or less, 110 nucleotides or less, 100 nucleotides or less, 95 nucleotides or less, 90 nucleotides or less, 85 nucleotides or less, 80 nucleotides or less, 75 nucleotides or less, 70 nucleotides or less, 65 nucleotides or less, 60 nucleotides or less, 55 nucleotides or less, 50 nucleotides or less, 45 nucleotides or less, 40 nucleotides or less, 35 nucleotides or less, 30 nucleotides or less, 25 nucleotides or less, 20 nucleotides or less, 15 nucleotides or less, 10 nucleotides or less, 9 nucleotides or less, 8 nucleotides or less, 7 nucleotides or less, 6 nucleotides or less, or 5 nucleotides or less from the first terminal repeat (e.g., ITR) or the second terminal repeat (e.g., ITR) of a vector construct disclosed herein.

In some aspects, the vector constructs provided herein comprise different nucleic acids that modulate a TLR. In some aspects, the vector constructs comprise one or more nucleic acids selected from SEQ ID NO: 1-38, SEQ ID NO: 54, SEQ ID NO: 55 or tandem repeats thereof, or any sequence of Table 1 or Table 2.

In some aspects, the nucleic acid that modulates a TLR comprises a sequence that has a stimulatory effect on a TLR (e.g., TLR9). In some aspects, the nucleic acid that modulates a TLR comprises a sequence that has an inhibitory effect on a TLR (e.g., TLR9). In some aspects, the nucleic acid that modulates a TLR comprises one or more tandem repeats. In some aspects, the nucleic acid that modulates a TLR comprises at least two tandem repeats of a sequence. In some aspects, the nucleic acid that modulates a TLR comprises at least two tandem repeats of a “TTAGGG” (SEQ ID NO: 55) sequence. In some aspects, the nucleic acid that modulates a TLR comprises SEQ ID NO: 3. In some aspects, the backbone polynucleotide comprise two or more copies of the nucleic acid that modulates a TLR. In some aspects, one or more of the copies of the nucleic acid that modulates a TLR (e.g., TLR9) are packaged in a viral particular (e.g., AAV).

In some aspects, the construct contains a promoter to facilitate expression of the nucleic acid of interest within a secretory cell. In some aspects, the promoter is a strong, eukaryotic promoter such as a promoter from cytomegalovirus (CMV), mouse mammary tumor virus (MMTV), Rous sarcoma virus (RSV), or adenovirus. Exemplary promoters include, but are not limited to the promoter from the immediate early gene of human CMV (Boshart et al., Cell 41:521-530 (1985) and the promoter from the long terminal repeat (LTR) of RSV (Gorman et al., Proc. Natl. Acad. Sci. USA 79:6777-6781 (1982)).

Alternatively, the promoter used can be a tissue-specific promoter. For example, where the secretory gland is a salivary gland, the tissue-specific promoter can be a salivary α-amylase promoter or mumps viral gene promoter and where the secretory gland is the pancreas, the promoter used in the vector can be a pancreas specific promoter, e.g., an insulin promoter or a pancreas α-amylase promoter. Both salivary and pancreatic α-amylase genes have been identified and characterized in both mice and humans (see e.g., Jones et al., Nucleic Acids Res., 17:6613-6623 (1989); Pittet et al., J. Mol. Biol., 182:359-365 (1985); Hagenbuchle et al., J. Mol. Biol., 185:285-293 (1985); Schibler et al., Oxf. Surv. Eukaryot. Genes, 3:210-234 (1986); and Sierra et al., Mol. Cell. Biol., 6:4067-4076 (1986) for murine pancreatic and salivary α-amylase genes and promoters; Samuelson et al., Nucleic Acids Res., 16:8261-8276 (1988); Groot et al., Genomics, 5:29-42 (1989); and Tomita et al., Gene, 76:11-18 (1989) for human pancreatic and salivary α-amylase genes and their promoters; Ting et al., Genes Dev. 6:1457-65 (1992) for human salivary α-amylase AMY1C promoter sequences).

In some aspects, the constructs of the disclosure can also include sequences in addition to promoters that enhance secretory gland specific expression. For example, where pancreas specific expression of the DNA of interest is desired, the construct can include a PTF-1 recognition sequence (Cockell et al., Mol. Cell. Biol., 9:2464-2476 (1989)). Sequences which enhance salivary gland specific expression are also well known in the art (see e.g., Robins et al., Genetica 86:191-201 (1992)).

In some aspects, the constructs of the disclosure can also include other components such as a marker (e.g., an antibiotic resistance gene (such as an ampicillin resistance gene) or (3-galactosidase) to aid in selection of cells containing and/or expressing the construct, an origin of replication for stable replication of the construct in a bacterial cell (preferably, a high copy number origin of replication), a nuclear localization signal, or other elements which facilitate production of the DNA construct, the protein encoded thereby, or both.

For eukaryotic expression, the construct can comprise at a minimum a eukaryotic promoter operably linked to a nucleic acid of interest, e.g, a DNA of interest, which is in turn operably linked to a polyadenylation sequence. The polyadenylation signal sequence can be selected from any of a variety of polyadenylation signal sequences known in the art. In some aspects, the polyadenylation signal sequence is the SV40 early polyadenylation signal sequence. The construct can also include one or more introns, which can increase levels of expression of the nucleic acid of interest, particularly where nucleic acid of interest is a DNA of interest and the DNA of interest is a cDNA (e.g., contains no introns of the naturally-occurring sequence). Any of a variety of introns known in the art can be used (e.g., the human β-globin intron, which is inserted in the construct at a position 5′ to the DNA of interest).

The nucleic acid of interest, e.g., DNA of interest, can be inserted into a construct so that the therapeutic molecule (e.g., a protein) is expressed as a fusion protein (e.g., a fusion protein having β-galactosidase or a portion thereof at the N-terminus and the therapeutic protein at the C-terminal portion). Production of a fusion protein can facilitate identification of transformed cells expressing the protein (e.g., by enzyme-linked immunosorbent assay (ELISA) using an antibody which binds to the fusion protein).

### III.A Delivery Vectors

In some aspects, the delivery vector is a viral vector, a non-viral vector, a plasmid, a lipid, or a lysosome.

### III.A.1 Non-Viral Vectors

The DNA of interest can be administered using a non-viral vector. “Non-viral vector,” as used herein is meant to include naked DNA, chemical formulations containing naked DNA (e.g., a formulation of DNA and cationic compounds (e.g., dextran sulfate)), and naked DNA mixed with an adjuvant such as a viral particle (i.e., the DNA of interest is not contained within the viral particle, but the transforming formulation is composed of both naked DNA and viral particles (e.g., AAV particles) (see e.g., Curiel et al., Am. J. Respir. Cell Mol. Biol. 6:247-52 (1992)). Thus the “non-viral vector” can include vectors composed of DNA plus viral particles where the viral particles do not contain the DNA of interest within the viral genome.

In some aspects, the non-viral vector is a bacterial vector. See e.g., Baban et al., Bioeng Bugs., 1(6):385-394 (2010).

In some aspects, the nucleic acid of interest, e.g., DNA of interest, can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the disclosure include DOPE (dioleyl phosphatidyl ethanol amine), CUDMEDA (N-(5-cholestrum-3(3-ol 3-urethanyl)-N′,N′-dimethylethylene diamine).

Lipids which can be used in accordance with the disclosure include, but are not limited to, DOPE (Dioleoyl phosphatidylethanolamine), cholesterol, and CUDMEDA (N-(5-cholestrum-3-ol 3 urethanyl)-N′,N′-dimethylethylenediamine). As an example, DNA can be administered in a solution containing one of the following cationic liposome formulations: Lipofectin™ (LTI/BRL), Transfast™ (Promega Corp), Tfx50™ (Promega Corp), Tfx10™ (Promega Corp), or Tfx20™ (Promega Corp). The concentration of the liposome solutions range from about 2.5% to 15% volume:volume, preferably about 6% to 12% volume:volume. Further exemplary methods and compositions for formulation of nucleic acid (e.g., DNA, including DNA or RNA not contained within a viral particle) for delivery according to the method of the disclosure are described in U.S. Pat. Nos. 5,892,071; 5,744,625; 5,925,623; 5,527,928; 5,824,812; 5,869,715.

Polymer particles can be used in accordance with the disclosure for polymer-based gene delivery. See e.g., Putnam et al., PNAS 98 (3): 1200-1205 (2001).

The nucleic acid of interest can also be administered as a chemical formulation of DNA or RNA coupled to a carrier molecule (e.g., an antibody or a receptor ligand) which facilitates delivery to host cells for the purpose of altering the biological properties of the host cells. The term “chemical formulations” refers to modifications of nucleic acids to allow coupling of the nucleic acid compounds to a carrier molecule such as a protein or lipid, or derivative thereof. Exemplary protein carrier molecules include antibodies specific to the cells of a targeted secretory gland or receptor ligands, i.e., molecules capable of interacting with receptors associated with a cell of a targeted secretory gland (e.g., salivary gland).

### III.A.2 Viral Vectors

In general, viral vectors used in accordance with the disclosure are composed of a viral particle derived from a naturally-occurring virus which has been genetically altered to render the virus replication-defective and to express a recombinant gene of interest in accordance with the disclosure. Once the virus delivers its genetic material to a cell, it does not generate additional infectious virus but does introduce exogenous recombinant genes into the cell, preferably into the genome of the cell.

Numerous viral vectors are well known in the art, including, for example, retrovirus, adenovirus, adeno-associated virus (AAV), herpes simplex virus (HSV), cytomegalovirus (CMV), vaccinia and poliovirus vectors. Retroviral vectors are less preferred since retroviruses require replicating cells and secretory glands are composed of mostly slowly replicating and/or terminally differentiated cells. Adenovirus and AAV are preferred viral vectors since this virus efficiently infects slowly replicating and/or terminally differentiated cells. In some aspects, the delivery vector (e.g., viral vector) is selected from the group consisting of an adeno-associated viral (AAV) vector, an adenoviral vector, a lentiviral vector, or a retroviral vector.

Where a replication-deficient virus is used as the viral vector, the production of infective virus particles containing either DNA or RNA corresponding to the nucleic acid of interest can be produced by introducing the viral construct into a recombinant cell line which provides the missing components essential for viral replication. In some aspects, transformation of the recombinant cell line with the recombinant viral vector will not result in production of replication-competent viruses, e.g., by homologous recombination of the viral sequences of the recombinant cell line into the introduced viral vector. Methods for production of replication-deficient viral particles containing a nucleic acid of interest are well known in the art and are described in, e.g., Rosenfeld et al., Science 252:431-434 (1991) and Rosenfeld et al., Cell 68:143-155 (1992) (adenovirus); U.S. Pat. No. 5,139,941 (adeno-associated virus); U.S. Pat. No. 4,861,719 (retrovirus); and U.S. Pat. No. 5,356,806 (vaccinia virus).

In certain aspects, a composition comprising a viral delivery vector comprising a nucleic acid of interest is suitable for delivery to a subject in need thereof. In certain aspects, a composition comprising a viral delivery vector comprising a nucleic acid of interest is suitable for delivery to a secretory organ. In some aspects, the secretory organ is selected from lymph node, gall bladder, thymus, hypothalamus, stomach, intestine, liver, pancreas, kidney, skin and/or secretory gland. In some aspects, the secretory organ is selected from heart, bone, muscle, skin, and/or adipose tissue. In some aspects, the secretory gland is a salivary gland, pancreas, a mammary gland, thyroid gland, parathyroid, an adrenal gland, a pineal body gland, thymus gland, pituitary gland, or hypothalamus. In some aspects, the secretory gland is a salivary gland.

### IV. Adeno-Associated Virus (AAV)-Mediated Gene Therapy

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

In some aspects, to produce recombinant AAV (rAAV) virions containing the a nucleic acid of interest, a suitable host cell line is transfected with a vector construct containing the a nucleic acid of interest, but lacking rep and cap. The host cell is then infected with wild-type (wt) AAV and a suitable helper virus to form rAAV virions. Alternatively, wt AAV genes (known as helper function genes, comprising rep and cap) and helper virus function genes (known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the heterologous gene. The heterologous gene is then replicated and packaged as though it were a wt AAV genome, forming a recombinant AAV virion. When a patient's cells are transduced with the resulting rAAV virion, the a nucleic acid of interest enters and is expressed in the patient's cells. Because the patient's cells lack the rep and cap genes, as well as the accessory function genes, the rAAV virion cannot further replicate and package its genomes. Moreover, without a source of rep and cap genes, wt AAV virions cannot be formed in the patient's cells. See e.g., U.S. Appl. Publ. No. 2003/0147853.

In some aspects, AAV vectors of the present disclosure can comprise or be derived from any natural or recombinant AAV serotype. According to the present disclosure, the AAV serotype can be, but is not limited to, AAV1, AAV2, AAV3, AAV4, AAV5, AAV6, AAV7, AAV8, AAVrh8, AAV9, AAV10, AAVrh10, AAV11, and AAV12. In some aspect, the AAV vector is modified relative to the wild-type AAV serotype sequence. In some aspects, the AAV serotype can be, but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, and AAVrh.74.

### IV.A. AAV Vector Components

### IV.A.1 Inverted Terminal Repeats (ITRs)

In some aspects, the AAV vectors of the present disclosure comprise a viral genome with at least one ITR region and a payload region, e.g., a nucleic acid of interest, e.g., a polynucleotide encoding a therapeutic protein, or a therapeutic peptide. In some aspects the AAV vector has two ITRs. These two ITRs flank the payload region e.g., a nucleic acid of interest, at the 5′ and 3′ ends. The ITRs function as origins of replication comprising recognition sites for replication. ITRs comprise sequence regions, which can be complementary and symmetrically arranged. ITRs incorporated into AAV vectors of the disclosure can be comprised of naturally occurring polynucleotide sequences or recombinantly derived polynucleotide sequences. In some aspects, the nucleic acid that modulates a TLR is positioned 500 bp or less, 450 bp or less, 400 bp or less, 350 bp or less, 300 bp or less, 250 bp or less, 200 bp or less, 150 bp or less, 100 bp or less, or 50 bp or less downstream or upstream from the 5′ or 3′ end of an ITR.

The ITRs can be derived from the same serotype as the capsid, selected from any of the serotypes listed herein, or a derivative thereof. The ITR can be of a different serotype from the capsid. In some aspects, the AAV vector has more than one ITR. In a non-limiting example, the AAV vector has a viral genome comprising two ITRs. In some aspects, the ITRs are of the same serotype as one another. In some aspects, the ITRs are of different serotypes. Non-limiting examples include zero, one or both of the ITRs having the same serotype as the capsid. In some aspects both ITRs of the AAV vector are AAV2 ITRs.

Independently, each ITR can be about 75 to about 175 nucleotides in length. An ITR can be about 100-105 nucleotides in length, about 106-110 nucleotides in length, about 111-115 nucleotides in length, about 116-120 nucleotides in length, about 121-125 nucleotides in length, about 126-130 nucleotides in length, about 131-135 nucleotides in length, about 136-140 nucleotides in length, about 141-145 nucleotides in length or about 146-150 nucleotides in length. In some aspects, the ITRs are about 140-142 nucleotides in length. Non-limiting examples of ITR length are about 102, about 140, about 141, about 142, about 145 nucleotides in length, and those having at least 95% identity thereto.

In some aspects, the vector construct and/or AAV vector comprises at least one inverted terminal repeat having a length such as, but not limited to, about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, or about 170-175 nucleotides.

In some aspects, the length of a first and/or a second ITR regions for the vector construct or AAV vector can be about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, and about 170-175 nucleotides.

In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located near the 5′ end of the flip ITR in the vector. In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide, which can be located near the 3′ end of the flip ITR in the vector. In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located near the 5′ end of the flop ITR in the vector. In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located near the 3′ end of the flop ITR in the vector. In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located between the 5′ end of the flip ITR and the 3′ end of the flop ITR in the vector. In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in the vector.

In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located within about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or more than about 30 nucleotides downstream or upstream from the 5′ or 3′ end of an ITR (e.g., Flip or Flop ITR) in the vector.

As another non-limiting example, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream or upstream from the 5′ or 3′ end of an ITR (e.g., Flip or Flop ITR) in the vector.

In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located within the first about 1%, about 2%, about 3%, about 4%, about 5%, about 6%, about 7%, about 8%, about 9%, about 10%, about 15%, about 20%, about 25% or more than about 25% of the nucleotides upstream from the 5′ or 3′ end of an ITR (e.g., Flip or Flop ITR) in the vector.

As another non-limiting example, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located with the first about 1-5%, about 1-10%, about 1-15%, about 1-20%, about 1-25%, about 5-10%, about 5-15%, about 5-20%, about 5-25%, about 10-15%, about 10-20%, about 10-25%, about 15-20%, about 15-25%, or about 20-25% downstream from the 5′ or 3′ end of an ITR (e.g., Flip or Flop ITR) in the vector.

### IV.A.2 Promoters

In some aspects, the payload region of the AAV vector comprises at least one element to enhance the nucleic acid specificity and/or expression. Non-limiting examples of elements to enhance the nucleic acid specificity and expression include, e.g., promoters, endogenous miRNAs, post-transcriptional regulatory elements (PREs), polyadenylation (Poly A) signal sequences and upstream enhancers (USEs), CMV enhancers, and introns.

Expression of a nucleic acid after delivery to or integration in the genomic DNA of a target cell can require a specific promoter, including but not limited to, a promoter that is species specific, inducible, tissue-specific, or cell cycle-specific (Parr et al., Nat. Med. 3: 1145-9 (1997); the contents of which are herein incorporated by reference in their entirety).

In some aspects, the promoter is deemed to be efficient when it drives expression of a nucleic acid of interest, e.g., one encoding a therapeutic protein, e.g., an antibody (e.g., a monoclonal antibody) or an antigen binding fragment thereof or a fusion protein (e.g., an Fc fusion protein), or therapeutic peptide carried in the payload region of the AAV vector. In some aspects, the promoter is a promoter deemed to be efficient when it drives expression of the therapeutic molecule of the present disclosure in the cell being targeted (e.g., secretory cell).

Promoters can be naturally occurring or non-naturally occurring. Non-limiting examples of promoters include viral promoters and mammalian promoters. In some aspects, the promoters can be human promoters. In some aspects, the promoter can be truncated. Promoters which drive or promote expression in most tissues include, but are not limited to, human elongation factor la-subunit (EF la), cytomegalovirus (CMV) immediate-early enhancer and/or promoter, chicken β-actin (CBA) and its derivative CAG, β glucuronidase (GUSB), or ubiquitin C (UBC). In some aspects, tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US 20110212529, the contents of which are herein incorporated by reference in their entirety). Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene ηβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some aspects, the promoter can be less than 1 kb. In some aspects, the promoter can have a length between about 15-20, about 10-50, about 20-30, about 30-40, about 40-50, about 50-60, about 50-100, about 60-70, about 70-80, about 80-90, about 90-100, about 100-110, about 100-150, about 110-120, about 120-130, about 130-140, about 140-150, about 150-160, about 150-200, about 160-170, about 170-180, about 180-190, about 190-200, about 200-210, about 200-250, about 210-220, about 220-230, about 230-240, about 240-250, about 250-260, about 250-300, about 260-270, about 270-280, about 280-290, about 290-300, about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides.

In some aspects, the promoter can be a combination of two or more components of the same or different starting or parental promoters such as, but not limited to, CMV and CBA.

In some aspects, each component in the promoter can have a length between about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides. In some aspects, the promoter is a combination of a 382 nucleotide CMV-enhancer sequence and a 260 nucleotide CBA-promoter sequence.

In some aspects, the AAV vector comprises a ubiquitous promoter. Non-limiting examples of ubiquitous promoters include, e.g., CMV, CBA (including derivatives CAG, CBh, etc.), EF-la, PGK, UBC, GUSB (hGBp), and UCOE (promoter of HNRPA2B1-CBX3).

In some aspects, the promoter is not cell specific. In some aspects, the promoter is a ubiquitin c (UBC) promoter. The UBC promoter can have a size of 300-350 nucleotides. In some aspects, the UBC promoter is 332 nucleotides. In some aspects, the promoter is a β-glucuronidase (GUSB) promoter. The GUSB promoter can have a size of 350-400 nucleotides. In some aspects, the GUSB promoter is 378 nucleotides. In some aspects, the promoter is a neurofilament light (NFL) promoter. The NFL promoter can have a size of 600-700 nucleotides. In some aspects, the NFL promoter is 650 nucleotides. In some aspects, the construct can be AAV-promoter-CMV/globin intron-modulatory polynucleotide-RBG, where the AAV can be self-complementary and the AAV can be the DJ serotype.

In some aspects, the AAV vector comprises a Pol III promoter. In some aspects, the AAV vector comprises a PI promoter. In some aspects, the AAV vector comprises a FXN promoter. In some aspects, the promoter is a phosphogly cerate kinase 1 (PGK) promoter. In some aspects, the promoter is a chicken β-actin (CBA) promoter. In some aspects, the promoter is a CAG promoter which is a construct comprising the cytomegalovirus (CMV) enhancer fused to the chicken beta-actin (CBA) promoter. In some aspects, the promoter is a cytomegalovirus (CMV) promoter. In some aspects, the AAV vector comprises a HI promoter. In some aspects, the AAV vector comprises a U6 promoter. In some aspects, the AAV vector comprises a SP6 promoter.

In some aspects, the promoter is a liver or a skeletal muscle promoter. Non-limiting examples of liver promoters include human a-1-antitrypsin (hAAT) and thyroxine binding globulin (TBG). Non-limiting examples of skeletal muscle promoters include Desmin, MCK or synthetic C5-12. In some aspects, the promoter is an RNA pol III promoter. In some aspects, the RNA pol III promoter is U6. In some aspects, the RNA pol III promoter is HI. In some aspects, the AAV vector comprises two promoters. In some aspects, the promoters are an EFla promoter and a CMV promoter.

In some aspects, the AAV vector comprises an enhancer element, a promoter and/or a 5′UTR intron. The enhancer element, also referred to herein as an “enhancer,” can be, but is not limited to, a CMV enhancer, the promoter can be, but is not limited to, a CMV, CBA, UBC, GUSB, NSE, Synapsin, MeCP2, and GFAP promoter and the 5′UTR/intron can be, but is not limited to, SV40, and CBA-MVM. In some aspects, the enhancer, promoter and/or intron used in combination can be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter, (9) GFAP promoter, (10) HI promoter; or (11) U6 promoter. In some aspects, the AA vector comprises an engineered promoter. In some aspects the AAV vector comprises a promoter from a naturally expressed protein.

### IV.A.3 Untranslated Regions (UTRs)

By definition, wild-type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs can be engineered into UTRs to enhance transcribed product stability and production. In some aspects, a 5′ UTR from mRNA normally expressed in the liver (e.g., albumin, serum amyloid A, Apolipoprotein AB/E, transferrin, alpha fetoprotein, erythropoietin, or Factor VIII) can be used in AAV vector of the disclosure to enhance expression, e.g., in brain tissue, and specifically in neuronal cells.

Wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG (SEQ ID NO: 48), where R is a purine (adenine or guanine) three bases upstream of the start codon (ATG), which is followed by another ′G. In some aspects, the 5′UTR in a AAV vector of the present disclosure includes a Kozak sequence. In some aspects, the 5′UTR in a vector construct or AAV vector of the present disclosure does not include a Kozak sequence.

Wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety). Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some aspects, microRNA targeting sequences are included to increase specificity of vector-mediated transgene expression. See, e.g., Anja Geisler and Henry Fechner, World J Exp Med., 20; 6(2):37-54 (2016).

In some aspects, the 3′ UTR of a vector construct or an AAV vector of the present disclosure can include an oligo(dT) sequence for addition of a poly-A tail. In some aspects, an AAV vector of the present disclosure can include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In some aspects, a vector construct or an AAV vector of the present disclosure can be engineered to include, alter or remove at least one miRNA binding site, sequence or seed region.

Any UTR from any gene known in the art can be incorporated into a vector construct or an AAV vector of the present disclosure. These UTRs, or portions thereof, can be placed in the same orientation as in the gene from which they were selected or they can be altered in orientation or location. In some aspects, the UTR used in a vector construct or an AAV vector of the present disclosure can be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR can be altered relative to a wild-type or native UTR by the change in orientation or location as taught above or can be altered by the inclusion of additional nucleotides, deletion of nucleotides, swapping or transposition of nucleotides. In some aspects, a vector construct or an AAV vector of the present disclosure comprises at least one artificial UTRs, which is not a variant of a wild-type UTR. In some aspects, a vector construct or an AAV vector of the present disclosure comprises UTRs which have been selected from a family of transcripts whose proteins share a common function, structure, feature or property.

### IV.A.4 Polyadenylation Sequence

In some aspects, the vector construct or AAV vectors of the present disclosure comprise at least one polyadenylation sequence. The vector construct or AAV vectors of the present disclosure can comprise a polyadenylation sequence between the 3′ end of the payload coding sequence and the 5′ end of the 3′ ITR.

In some aspects, the polyadenylation sequence or “polyA sequence” can range from absent to about 500 nucleotides in length.

In some aspects, the polyadenylation sequence is about 50-100, about 50-150, about 50-160, about 50-200, about 60-100, about 60-150, about 60-160, about 60-200, about 70-100, about 70-150, about 70-160, about 70-200, about 80-100, about 80-150, about 80-160, about 80-200, about 90-100, about 90-150, about 90-160, or about 90-200 nucleotides in length.

In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located upstream of the polyadenylation sequence in the vector. In some aspects, the vector construct or AAV vector comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located downstream of a promoter such as, but not limited to, CMV, U6, CAG, CBA or a CBA promoter with a SV40 intron or a human betaglobin intron in the vector.

In some aspects, the vector construct or AAV vector of the present disclosure comprises a nucleic acid sequence of interest, e.g., one encoding a therapeutic protein, such as an antibody (e.g., a monoclonal antibody) or an antigen binding fragment thereof or a fusion protein (e.g., an Fc fusion protein), or a therapeutic peptide which can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream from the promoter and/or upstream of the polyadenylation sequence in the vector.

In some aspects, the vector construct or AAV vector comprises a rabbit globin polyadenylation (poly A) signal sequence. In some aspects, the vector construct or AAV vector comprises a human growth hormone polyadenylation (poly A) signal sequence. In some aspects, the vector construct or AAV vector comprises a bovine growth hormone polyadenylation (poly A) signal sequence.

### IV.A.5 Introns

In some aspects, the payload region of a vector construct or an AAV vector of the present disclosure comprises at least one element to enhance the expression such as one or more introns or portions thereof. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some aspects, the intron or intron portion can be between about 100 and about 500 nucleotides in length. In some aspects, the intron can have a length between about 80-100, about 80-120, about 80-140, about 80-160, about 80-180, about 80-200, about 80-250, about 80-300, about 80-350, about 80-400, about 80-450, about 80-500, about 200-300, about 200-400, about 200-500, about 300-400, about 300-500, or about 400-500 nucleotides.

In some aspects, the vector construct or AAV vector can comprise a promoter such as, but not limited to, CMV or U6. In some aspects, the promoter for an AAV vector of the present disclosure is a CMV promoter. As another non-limiting example, the promoter for an AAV vector of the present disclosure is a U6 promoter. In some aspects, the vector construct or AAV vector can comprise a CMV and a U6 promoter. In some aspects, the vector construct or AAV vector can comprise a HI promoter. In some aspects, the vector construct or AAV vector can comprise a CBA promoter. In some aspects, the vector construct or AAV vector can comprise a chimeric intron.

In some aspects, a therapeutic protein encoded by a nucleic acid of interest, e.g., antibody (e.g., a monoclonal antibody) or antigen binding fragment thereof or the fusion protein (e.g., the Fc fusion protein), or therapeutic peptide can be located downstream of a promoter in an expression vector such as, but not limited to, CMV, U6, HI, CBA, CAG, or a CBA promoter with an intron such as SV40 or others known in the art. Further, the nucleic acid of interest can also be located upstream of the polyadenylation sequence in an expression vector. In some aspects, the nucleic acid of interest can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream from the promoter and/or upstream of the polyadenylation sequence in the vector.

### IV.A.6 Filler Sequences

In some aspects, the vector construct, backbone, or AAV vector comprises one or more filler sequences (also referred to as “stuffer sequences”). In some aspects, the vector construct or backbone comprises one or more filler sequences in order to have the length of the vector construct or backbone be the optimal size for packaging. In some aspects, the vector construct or backbone comprises at least one filler sequence in order to have the length of the vector construct or backbone be about 2.0-2.5 kb, e.g., about 2.3 kb. In some aspects, the vector construct or backbone comprises at least one filler sequence in order to have the length of the vector construct or backbone be about 4.6 kb.

In some aspects, the vector construct or backbone comprises one or more filler sequences in order to reduce the likelihood that a hairpin structure of the vector genome (e.g., a modulatory polynucleotide described herein) can be read as an inverted terminal repeat (ITR) during expression and/or packaging. In some aspects, the vector construct or backbone comprises at least one filler sequence in order to have the length of the vector construct or backbone be about 2.0-2.5 kb, e.g., about 2.3 kb. In some aspects, the vector construct or backbone comprises at least one filler sequence in order to have the length of the vector construct or backbone be about 4.6 kb.

In some aspects, the vector is a single stranded (ss) vector and comprises one or more filler sequences which have a length about between 0.1 kb and about 3.8 kb, such as, but not limited to, about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3 kb, about 3.1 kb, about 3.2 kb, about 3.3 kb, about 3.4 kb, about 3.5 kb, about 3.6 kb, about 3.7 kb, or about 3.8 kb.

In some aspects, the vector is a self-complementary (sc) vector and comprises one or more filler sequences which have a length about between about 0.1 kb and about 1.5 kb, such as, but not limited to, about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.1 kb, about 1.2 kb, about 1.3 kb, about 1.4 kb, or about 1.5 kb.

In some aspects, the vector comprises any portion of a filler sequence. The vector can comprise, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of a filler sequence.

In some aspects, the vector is a single stranded (ss) vector and comprises one or more filler sequences in order to have the length of the vector be about 4.6 kb. In some aspects, the vector comprises at least one filler sequence and the filler sequence is located 3′ to the 5′ ITR sequence. In some aspects, the vector comprises at least one filler sequence and the filler sequence is located 5′ to a promoter sequence. In some aspects, the vector comprises at least one filler sequence and the filler sequence is located 3′ to the polyadenylation signal sequence. In some aspects, the vector comprises at least one filler sequence and the filler sequence is located 5′ to the 3′ ITR sequence. In some aspects, the vector comprises at least one filler sequence, and the filler sequence is located between two intron sequences. In some aspects, the vector comprises at least one filler sequence, and the filler sequence is located within an intron sequence. In some aspects, the vector comprises two filler sequences, and the first filler sequence is located 3′ to the 5′ ITR sequence and the second filler sequence is located 3′ to the polyadenylation signal sequence. In some aspects, the vector comprises two filler sequences, and the first filler sequence is located 5′ to a promoter sequence and the second filler sequence is located 3′ to the polyadenylation signal sequence. In some aspects, the vector comprises two filler sequences, and the first filler sequence is located 3′ to the 5′ ITR sequence and the second filler sequence is located 5′ to the 5′ ITR sequence.

In some aspects, the vector is a self-complementary (sc) vector and comprises one or more filler sequences in order to have the length of the vector be about 2.3 kb. In some aspects, the vector comprises at least one filler sequence and the filler sequence is located 3′ to the 5′ ITR sequence. In some aspects, the vector comprises at least one filler sequence and the filler sequence is located 5′ to a promoter sequence. In some aspects, the vector comprises at least one filler sequence and the filler sequence is located 3′ to the polyadenylation signal sequence. In some aspects, the vector comprises at least one filler sequence and the filler sequence is located 5′ to the 3′ ITR sequence.

In some aspects, the vector comprises at least one filler sequence, and the filler sequence is located between two intron sequences. In some aspects, the vector comprises at least one filler sequence, and the filler sequence is located within an intron sequence. In some aspects, the vector comprises two filler sequences, and the first filler sequence is located 3′ to the 5′ ITR sequence and the second filler sequence is located 3′ to the polyadenylation signal sequence. In some aspects, the vector comprises two filler sequences, and the first filler sequence is located 5′ to a promoter sequence and the second filler sequence is located 3′ to the polyadenylation signal sequence. In some aspects, the vector comprises two filler sequences, and the first filler sequence is located 3′ to the 5′ ITR sequence and the second filler sequence is located 5′ to the 5′ ITR sequence.

In some aspects, the vector can comprise one or more filler sequences between one of more regions of the vector. In some aspects, the filler region can be located before a region such as, but not limited to, a payload region, an ITR, a promoter region, an intron region, an enhancer region, and/or a polyadenylation signal sequence region. In some aspects, the filler region can be located after a region such as, but not limited to, a payload region, an ITR, a promoter region, an intron region, an enhancer region, and/or a polyadenylation signal sequence region. In some aspects, the filler region can be located before and after a region such as, but not limited to, a payload region, an ITR, a promoter region, an intron region, an enhancer region, and/or a polyadenylation signal sequence region.

In some aspects, the vector can comprise one or more filler sequences which bifurcates at least one region of the vector. The bifurcated region of the vector can comprise about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the of the region to the 5′ of the filler sequence region.

In some aspects, the filler sequence can bifurcate at least one region so that about 10% of the region is located 5′ to the filler sequence and about 90% of the region is located 3′ to the filler sequence. In some aspects, the filler sequence can bifurcate at least one region so that about 20% of the region is located 5′ to the filler sequence and about 80% of the region is located 3′ to the filler sequence. In some aspects, the filler sequence can bifurcate at least one region so that about 30% of the region is located 5′ to the filler sequence and about 70% of the region is located 3′ to the filler sequence. In some aspects, the filler sequence can bifurcate at least one region so that about 40% of the region is located 5′ to the filler sequence and about 60% of the region is located 3′ to the filler sequence. In some aspects, the filler sequence can bifurcate at least one region so that about 50% of the region is located 5′ to the filler sequence and about 50% of the region is located 3′ to the filler sequence. In some aspects, the filler sequence can bifurcate at least one region so that about 60% of the region is located 5′ to the filler sequence and about 40% of the region is located 3′ to the filler sequence. In some aspects, the filler sequence can bifurcate at least one region so that about 70% of the region is located 5′ to the filler sequence and about 30% of the region is located 3′ to the filler sequence. In some aspects, the filler sequence can bifurcate at least one region so that about 80% of the region is located 5′ to the filler sequence and about 20% of the region is located 3′ to the filler sequence. In some aspects, the filler sequence can bifurcate at least one region so that about 90% of the region is located 5′ to the filler sequence and about 10% of the region is located 3′ to the filler sequence.

In some aspects, the vector comprises a filler sequence after the 5′ ITR. In some aspects, the AAV vector comprises a filler sequence after the promoter region. In some aspects, the vector comprises a filler sequence after the payload region. In some aspects, the vector comprises a filler sequence after the intron region. In some aspects, the vector comprises a filler sequence after the enhancer region. In some aspects, the vector comprises a filler sequence after the polyadenylation signal sequence region. In some aspects, the vector comprises a filler sequence before the promoter region. In some aspects, the vector comprises a filler sequence before the payload region. In some aspects, the vector comprises a filler sequence before the intron region.

In some aspects, the vector comprises a filler sequence before the enhancer region. In some aspects, the vector comprises a filler sequence before the polyadenylation signal sequence region. In some aspects, the vector comprises a filler sequence before the 3′ ITR. In some aspects, a filler sequence can be located between two regions, such as, but not limited to, the 5′ ITR and the promoter region. In some aspects, a filler sequence can be located between two regions, such as, but not limited to, the 5′ ITR and the payload region.

In some aspects, a filler sequence can be located between two regions, such as, but not limited to, the 5′ ITR and the intron region. In some aspects, a filler sequence can be located between two regions, such as, but not limited to, the 5′ ITR and the enhancer region. In some aspects, a filler sequence can be located between two regions, such as, but not limited to, the 5′ ITR and the polyadenylation signal sequence region. In some aspects, a filler sequence can be located between two regions, such as, but not limited to, the promoter region and the payload region.

In some aspects, a filler sequence can be located between two regions, such as, but not limited to, the promoter region and the intron region. In some aspects, a filler sequence can be located between two regions, such as, but not limited to, the promoter region and the enhancer region. In some aspects, a filler sequence can be located between two regions, such as, but not limited to, the promoter region and the polyadenylation signal sequence region. In some aspects, a filler sequence can be located between two regions, such as, but not limited to, the promoter region and the 3′ ITR.

In some aspects, a filler sequence can be located between two regions, such as, but not limited to, the payload region and the intron region. In some aspects, a filler sequence can be located between two regions, such as, but not limited to, the payload region and the enhancer region. In some aspects, a filler sequence can be located between two regions, such as, but not limited to, the payload region and the polyadenylation signal sequence region. In some aspects, a filler sequence can be located between two regions, such as, but not limited to, the payload region and the 3′ ITR.

In some aspects, a filler sequence can be located between two regions, such as, but not limited to, the intron region and the enhancer region. In some aspects, a filler sequence can be located between two regions, such as, but not limited to, the intron region and the polyadenylation signal sequence region. In some aspects, a filler sequence can be located between two regions, such as, but not limited to, the intron region and the 3′ ITR. In some aspects, a filler sequence can be located between two regions, such as, but not limited to, the enhancer region and the polyadenylation signal sequence region. In some aspects, a filler sequence can be located between two regions, such as, but not limited to, the enhancer region and the 3′ ITR. In some aspects, a filler sequence can be located between two regions, such as, but not limited to, the polyadenylation signal sequence region and the 3′ ITR.

In some aspects, a vector can comprise two filler sequences. The two filler sequences can be located between two regions as described herein.

### IV.A.7 AAV Capsids

A wild-type adeno-associated virus (AAV) particle contains a single-stranded DNA genome and a capsid comprised of three structural Cap proteins (VP1, -2 and -3). The majority of its T ¼ 1 icosahedral architecture is made up by the 60 kDa VP3 proteins. For every 10 VP3 molecules, one VP1 and one VP2 capsid monomer is anchored in the particle structure by the C terminus that is structurally identical to the VP3 monomer.

The capsid is the primary interface between the host and the vector genome, and is implicated in determining cell and tissue tropism. For example, in several animal models, AAV4 targets the retinal pigment epithelium following subretinal delivery more specifically than AAV2. In the mouse lung, AAV5 and AAV6 target cells primarily of the conducting airway, whereas AAV9 has a more alveolar tropism.

In some aspects, an AAV vector or AAV particle of the disclosure comprises an AAV capsid encapsulating a nucleic acid sequence of interest and a nucleic acid sequence that modulates the TLR. In some aspects, the nucleic acid sequence that modulates the TLR comprises about 0.01% to about 5%, about 0.01% to about 4%, about 0.01% to about 3%, about 0.01% to about 2%, about 0.01% to about 1%, about 0.01% to about 0.5%, about 0.05% to about 5%, about 0.05% to about 4%, about 0.05% to about 3%, about 0.05% to about 2%, about 0.05% to about 1%, about 0.05% to about 0.5%, about 0.1% to about 5%, about 0.1% to about 4%, about 0.1% to about 3%, about 0.1% to about 2%, about 0.1% to about 1%, about 0.1% to about 0.5%, about 0.5% to about 5%, about 0.5% to about 4%, about 0.5% to about 3%, about 0.5% to about 2%, or about 0.5% to about 1% of the encapsulated nucleic acid sequence.

IV.A.8 Packaging of Nucleic Acids into AAV Capsids

In some aspects, both ends of the AAV genome contain inverted terminal repeats (ITRs) of approximately 150 nucleotides (NTs) that form T-shaped hairpin secondary structures. ITRs are sectioned into four regions that contain cis-elements, including the Rep binding element (RBE) and the terminal resolution site (trs), which are indispensable for genome replication and packaging. The ITRs flank two large open reading frames (ORFs) which code for a series of replication (Rep) proteins and viral proteins (VPs), and the assembly-activating protein.

The AAV packaging process occurs in the nucleus of the cell producing the AAV capsid and relies on the presence of: assembled empty capsids, replicated AAV genomes and the viral Rep proteins. For the packaging of a certain DNA molecule into AAV capsids, the wild-type (WT) AAV genome or any transgene cassette, the DNA sequence must be flanked by the cis-acting ITRs. These not only represent the packaging signals but also aid the amplification of the DNA genome by a self-primed replication mechanism. The products of the replication are DNA genomes of positive and negative polarity that are packaged into the AAV virions with equal frequency.

The current model for AAV packaging couples the replication and packaging process. This is supported by the observation that the Rep proteins, capsids and the AAV DNA colocalize in the nucleoplasm in infected cells. During AAV replication, the N-terminus of the large Rep proteins bind specifically to the RBE within the ITRs and cleave the trs which allows self-priming and induces further replication.

Eventually the displacement of the ssDNA genome occurs due to DNA replication of the complementary strand. In that process, the single-stranded binding proteins, E2A (adenovirus) or ICP8 (herpes virus) provided by the helper viruses are involved.

After one single-stranded genome is fully displaced, the DNA is reported to be translocated into the capsid in a 3′ to 5′ direction by the helicase/ATPase domain of the small Rep proteins. The translocation of the ssDNA into the capsid is believed to occur through the channel at the fivefold symmetry axis.

While the large Rep proteins (Rep78/68) are reported to bind to the ITRs, the small Rep proteins (Rep52/40) are reported to interact with ssDNA in the nucleus, with the affinity of the former interaction being greater than the latter. The absence of the small Rep proteins, a mutation in the helicase/ATPase domain or a capsid-binding deficient Rep52/40 variant leads to significantly reduced amounts of genome-containing AAV particles. These observations support a central role for the small Rep proteins in AAV genome packaging. With respect to the role of the capsid in this process, mutations of residues assembling the channel at the fivefold, for example T329/T330A, in the AAV2 capsid show a severe reduction of Rep-capsid interaction and genome packaging.

### IV.A.9 Methods for Producing Recombinant AAVs

In some aspects, the present disclosure provides methods for the generation of AAV particles, by viral genome replication in a viral replication cell comprising contacting the viral replication cell with an AAV polynucleotide or AAV genome. In the context of the present disclosure, the AAV vectors disclosed herein can comprising a polynucleotide of interest, ITRs, and a polynucleotide that modulates a TLR derived from a vector construct disclosed herein.

In some aspects, an AAV particle is produced by a method comprising the steps of: (1) co-transfecting competent bacterial cells with a bacmid vector and either a viral construct vector and/or AAV payload construct vector, (2) isolating the resultant viral construct expression vector and AAV payload construct expression vector and separately transfecting viral replication cells, (3) isolating and purifying resultant payload and viral construct particles comprising viral construct expression vector or AAV payload construct expression vector, (4) co-infecting a viral replication cell with both the AAV payload and viral construct particles comprising viral construct expression vector or AAV payload construct expression vector, and (5) harvesting and purifying the viral particle comprising a parvoviral genome.

In some aspect, the present disclosure provides a method for producing an AAV particle comprising the steps of (1) simultaneously co-transfecting mammalian cells, such as, but not limited to HEK293 cells, with a payload region (e.g., nucleic acid of interest), a construct expressing rep and cap genes and a helper construct, and (2) harvesting and purifying the AAV particle comprising a viral genome.

In some aspects, the AAV particles can be produced in a viral replication cell that comprises an insect cell. Growing conditions for insect cells in culture, and production of heterologous products in insect cells in culture are well-known in the art, see, e.g., U.S. Pat. No. 6,204,059.

The viral replication cell can be selected from any biological organism, including prokaryotic (e.g., bacterial) cells, and eukaryotic cells, including, insect cells, yeast cells and mammalian cells. Viral replication cells can comprise mammalian cells such as A549, WEH1, 3T3, 10T1/2, BHK, MDCK, COS 1, COS 7, BSC 1, BSC 40, BMT 10, VERO. W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

Viral production disclosed herein describes processes and methods for producing AAV particles that contact a target cell to deliver a payload, e.g. a recombinant viral construct, which comprises a polynucleotide of interest.

In some aspects, the AAV particles can be produced in a viral replication cell that comprises a mammalian cell. Viral replication cells commonly used for production of recombinant AAV particles include, but are not limited to 293 cells, COS cells, HeLa cells, and KB cells.

In some aspects, AAV particles are produced in mammalian cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In some aspects, AAV particles are produced in mammalian cells using a triple transfection method wherein a payload construct, parvoviral Rep and parvoviral Cap and a helper construct are comprised within three different constructs. The triple transfection method of the three components of AAV particle production can be utilized to produce small lots of virus for assays including transduction efficiency, target tissue (tropism) evaluation, and stability.

In some aspects, the viral construct vector and the AAV payload construct vector can be each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses can be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see, e.g., Urabe, M. et al., J Virol. 2006 February; 80 (4): 1874-85, the contents of which are herein incorporated by reference in their entirety.

Production of AAV particles with baculovirus in an insect cell system can address known baculovirus genetic and physical instability. Baculovirus-infected viral producing cells are harvested into aliquots that can be cryopreserved in liquid nitrogen; the aliquots retain viability and infectivity for infection of large-scale viral producing cell culture (Wasilko D J et al., Protein Expr Purif. 2009 June; 65(2): 122-32).

In some aspects, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some aspects, AAV particle production can be modified to increase the scale of production. Transfection of replication cells in large-scale culture formats can be carried out according to any methods known in the art.

In some aspects, cell culture bioreactors can be used for large scale viral production. In some cases, bioreactors comprise stirred tank reactors.

### IV.A.10 Cell Lysis

Cells of the disclosure, including, but not limited to viral production cells, can be subjected to cell lysis according to any methods known in the art. Cell lysis can be carried out to obtain one or more agents (e.g. viral particles) present within any cells of the disclosure.

Cell lysis methods can be chemical or mechanical. Chemical cell lysis typically comprises contacting one or more cells with one or more lysis agent. Mechanical lysis typically comprises subjecting one or more cells to one or more lysis condition and/or one or more lysis force. In some aspects, chemical lysis can be used to lyse cells. As used herein, the term “lysis agent” refers to any agent that can aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions can include one or more buffering agents, solubilizing agents, surfactants, preservatives, cryoprotectants, enzymes, enzyme inhibitors and/or chelators.

Concentrations of salts can be increased or decreased to obtain an effective concentration for rupture of cell membranes. Lysis agents comprising detergents can include ionic detergents or non-ionic detergents. Detergents can function to break apart or dissolve cell structures including, but not limited to cell membranes, cell walls, lipids, carbohydrates, lipoproteins and glycoproteins.

In some aspects, mechanical cell lysis is carried out. Mechanical cell lysis methods can include the use of one or more lysis condition and/or one or more lysis force. As used herein, the term “lysis condition” refers to a state or circumstance that promotes cellular disruption. Lysis conditions can comprise certain temperatures, pressures, osmotic purity, salinity and the like. In some aspects, lysis conditions comprise increased or decreased temperatures. In some aspects, lysis conditions comprise changes in temperature to promote cellular disruption. Cell lysis carried out according to such aspects can include freeze-thaw lysis.

As used herein, the term “lysis force” refers to a physical activity used to disrupt a cell. Lysis forces can include, but are not limited to mechanical forces, sonic forces, gravitational forces, optical forces, electrical forces and the like. Cell lysis carried out by mechanical force is referred to herein as “mechanical lysis.” Mechanical forces that can be used according to mechanical lysis can include high shear fluid forces.

In some aspects, a method for harvesting AAV particles without lysis can be used for efficient and scalable AAV particle production. In a non-limiting example, AAV particles can be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107.

### IV.A.11 AAV Purification

Cell lysates comprising viral particles can be subjected to clarification. Clarification refers to initial steps taken in purification of viral particles from cell lysates. Clarification serves to prepare lysates for further purification by removing larger, insoluble debris. Clarification steps can include, but are not limited to centrifugation and filtration.

In some aspects, AAV particles can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography), immunoaffinity chromatography and size-exclusion chromatography.

### V. Methods of Treatment and Use

Some aspects of the present disclosure generally relate to a method of modulating an immune response in a subject, comprising administering to said subject an effective amount of the AAV particle as described herein, e.g., one comprising a polynucleotide that modulates a TLR. In some aspects, In some aspects, the response includes but is not limited to: preventing and/or reducing TLR9-mediated inflammation; reducing induction of pro-inflammatory cytokines; and increasing nucleic acid of interest expression. In some aspects, the response includes, but is not limited to, responses such as stimulating monocytes, macrophages, and dendritic cells that then produce several cytokines, including the TH1 cytokine interleukin 12.

In some aspects, the nucleic acid sequence that modulates the TLR is capable of inhibiting a TLR inflammatory response. In some aspects, the nucleic acid sequence that modulates a TLR reduces the subject's immune response to a gene therapy. In some aspects, the nucleic acid sequence that modulates a TLR is capable of activating an inflammatory response. In some aspects, the nucleic acid that modulates a TLR enhances the subject's immune response to a tumor.

Some aspects of the present disclosure relate to a method of reducing immunogenicity of an AAV capsid comprising packaging a portion of a backbone polynucleotide comprising a nucleic acid sequence that modulates a Toll-like receptor (TLR) into the AAV capsid, whereby the AAV capsid causes a reduced inflammatory response in a host as compared to an AAV capsid that does not contain the portion of a backbone comprising the nucleic acid sequence that modulates the TLR.

Some aspects of the present disclosure relate to a method of enhancing immunogenicity of an AAV capsid comprising packaging a portion of a backbone polynucleotide comprising a nucleic acid sequence that modulates a Toll-like receptor (TLR) into the AAV capsid, whereby the AAV capsid causes an enhanced inflammatory response in a host as compared to an AAV capsid that does not contain the portion of a backbone comprising the nucleic acid sequence that modulates the TLR. In some aspects, the AAV capsid is an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12. In some aspects, the AAV capsid comprises about 0.01% to about 2% of the polynucleotide backbone sequence. In some aspects, the AAV capsid comprises about 0.01% to about 1% of the polynucleotide backbone sequence. In some aspects, the TLR comprise a TLR3, a TLR4, a TLR7, a TLR8, a TLR9, or any combination thereof. In some aspects, the TLR comprises TLR9. In some aspects, the backbone polynucleotide comprises two or more copies of the nucleic acid sequences that modulates TLR9. In some aspects, the nucleic acid sequence that modulates the TLR comprises a sequence selected from any of SEQ ID NOs: 1-38, or any combination thereof.

### VI. Pharmaceutical Compositions

In some aspects, a pharmaceutical composition disclosed herein comprises a viral vectors (e.g., AAV vector) or AAV particles of the present disclosure and a pharmaceutically-acceptable excipient or carrier in a form suitable for administration to a subject. Pharmaceutically acceptable excipients or carriers are determined in part by the particular composition being administered, as well as by the particular method used to administer the composition.

Accordingly, there is a wide variety of suitable formulations of pharmaceutical compositions comprising a construct of the present disclosure or a plurality thereof (see, e.g., Remington's Pharmaceutical Sciences, Mack Publishing Co., Easton, Pa. 18th ed. (1990)) and/or one or more shRNA disclosed herein. The pharmaceutical compositions are generally formulated sterile and in full compliance with all Good Manufacturing Practice (GMP) regulations of the U.S. Food and Drug Administration.

In some aspects, a pharmaceutical composition comprises (i) one or more constructs disclosed herein, and (ii) one or more therapeutic agents for the treatment of a disorder. In some aspects, the one or more viral vectors (e.g., AAV vector) or AAV particles disclosed herein and the one or more therapeutic agents for a disease or disorder (e.g., an immune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder, an inflammatory disease or disorder, a cardiovascular disease or disorder, a gastrointestinal disease or disorder, a pulmonary disease or disorder, a rheumatological disease or disorder, a parasitic infection, a fungal infection (e.g., an infection by a spore-forming fungi), a bacterial infection (e.g., an infection by a spore-forming bacteria), a viral infection, and any combination thereof) are co-administered in a single pharmaceutical composition. In some aspects, the one or more viral vectors (e.g., AAV vector) or AAV particles disclosed herein and the one or more therapeutic agents for the treatment of a disease or disorder (e.g., an immune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder, an inflammatory disease or disorder, a cardiovascular disease or disorder, a gastrointestinal disease or disorder, a pulmonary disease or disorder, a rheumatological disease or disorder, a parasitic infection, a fungal infection (e.g., an infection by a spore-forming fungi), a bacterial infection (e.g., an infection by a spore-forming bacteria), a viral infection, and any combination thereof) are co-administered as separate pharmaceutical compositions.

In some aspects, a pharmaceutical composition comprising one or more viral vectors (e.g., AAV vector) or AAV particles disclosed herein is administered prior to the administration of a pharmaceutical composition comprising one or more therapeutic agents for the treatment of a disease or disorder (e.g., an immune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder, an inflammatory disease or disorder, a cardiovascular disease or disorder, a gastrointestinal disease or disorder, a pulmonary disease or disorder, a rheumatological disease or disorder, a parasitic infection, a fungal infection (e.g., an infection by a spore-forming fungi), a bacterial infection (e.g., an infection by a spore-forming bacteria), a viral infection, and any combination thereof). In some aspects, a pharmaceutical composition comprising one or more viral vectors (e.g., AAV vector) or AAV particles disclosed herein is administered after the administration of a pharmaceutical composition comprising one or more therapeutic agents for the treatment of a disease or disorder (e.g., an immune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder, an inflammatory disease or disorder, a cardiovascular disease or disorder, a gastrointestinal disease or disorder, a pulmonary disease or disorder, a rheumatological disease or disorder, a parasitic infection, a fungal infection (e.g., an infection by a spore-forming fungi), a bacterial infection (e.g., an infection by a spore-forming bacteria), a viral infection, and any combination thereof). In some aspects, a pharmaceutical composition comprising one or more viral vectors (e.g., AAV vector) or AAV particles disclosed herein is administered concurrently with a pharmaceutical composition comprising one or more therapeutic agents for the treatment of a disease or disorder (e.g., an immune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder, an inflammatory disease or disorder, a cardiovascular disease or disorder, a gastrointestinal disease or disorder, a pulmonary disease or disorder, a rheumatological disease or disorder, a parasitic infection, a fungal infection (e.g., an infection by a spore-forming fungi), a bacterial infection (e.g., an infection by a spore-forming bacteria), a viral infection, and any combination thereof).

In some aspects, the pharmaceutical composition of the disclosure is formulated for intramuscular, intraveneous, intratumoral, or intraductal administration. In some aspects, the pharmaceutical composition of the disclosure is formulated for direct injection.

Also provided herein are pharmaceutical compositions comprising viral vectors (e.g., AAV vector) or AAV particles disclosed herein having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of vectors, e.g., AAV vectors described herein. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

Acceptable carriers, excipients, or stabilizers are nontoxic to recipients (e.g., animals or humans) at the dosages and concentrations employed.

Examples of carriers or diluents include, but are not limited to, water, saline, Ringer's solutions, dextrose solution, and 5% human serum albumin. Except insofar as any conventional media or compound is incompatible with the delivery vectors disclosed herein (e.g., viral vectors (e.g., AAV vector) or AAV particles), use thereof in the compositions is contemplated. In some aspects, a pharmaceutical composition is formulated to be compatible with its intended route of administration. The delivery vectors disclosed herein (e.g., viral vectors (e.g., AAV vector) or AAV particles) can be administered by parenteral, topical, intravenous, oral, subcutaneous, intra-arterial, intradermal, transdermal, rectal, intracranial, intraperitoneal, intranasal, intratumoral, intramuscular route or as inhalants. In some aspects, the pharmaceutical composition comprising the delivery vectors disclosed herein (e.g., viral vectors (e.g., AAV vector) or AAV particles) is administered intravenously, e.g. by injection. In some aspects, the pharmaceutical composition comprising the delivery vectors disclosed herein (e.g., viral vectors (e.g., AAV vector) or AAV particles) is administered intramuscularly. The delivery vectors disclosed herein (e.g., viral vectors (e.g., AAV vector) or AAV particles) can optionally be administered in combination with other therapeutic agents that are at least partly effective in treating the disease, disorder or condition for which the delivery vectors disclosed herein (e.g., viral vectors (e.g., AAV vector) or AAV particles) are intended.

The constructs disclosed herein can be formulated using one or more excipients to (1) increase stability; (2) increase cell transfection or transduction; (3) permit the sustained or delayed release; or (4) alter the biodistribution (e.g., target the viral vectors (e.g., AAV vector) or AAV particles to specific tissues or cell types such as secretory cells).

### VII. Administration

The gene therapy compositions, constructs, and delivery vectors disclosed herein (e.g., viral vectors (e.g., AAV vector) or AAV particles) can be administered by any route which results in a therapeutically effective outcome. In some aspects, the delivery can be intramuscular (IM), intravenous (IV), intraductal, or direct injection to a secretory organ or a secretory-like organ.

In some aspects, compositions of viral vectors (e.g., AAV vector) or AAV particles disclosed herein can be administered in a way which facilitates the vectors to enter a secretary organ of the subject. In some aspects, the secretory organ is selected from lymph node, gall bladder, thymus, hypothalamus, stomach, intestine, liver, pancreas, kidney, skin and/or secretory gland. In some aspects, the secretory organ is selected from heart, bone, muscle, skin, and/or adipose tissue. In some aspects, the muscle is skeletal muscle.

In certain aspects, the administration is intraductal. In some aspects, the constructs described herein are introduced into the secretory organ (e.g., a secretory gland) in vivo via the duct system (e.g., by retrograde intraductal administration, which can be accomplished by perfusion (e.g., continuous injection), or by a single, discontinuous injection). Intraductal administration can also be accomplished by cannulation, which can be accomplished for the pancreas and the liver by, for example, insertion of the cannula through a lumen of the gastrointestinal tract, by insertion of the cannula through an external orifice, insertion of the cannula through the common bile duct. Retrograde ductal administration can be accomplished in the pancreas and liver by endoscopic retrograde chalangio-pancreatography (ECRP). The methods of the disclosure can involve delivery to the pancreas, the liver, the salivary gland, or to any combination thereof. In some aspects, ductal administration provides advantages, e.g., because the vector is presented to the cells from “outside” the body (from the lumen), the immunological and inflammatory reactions that are commonly observed as a result of the administration of transforming formulations and their adjuvants into blood and interstitial fluid can be avoided.

Moreover, the cells of secretory glands form a monolayer that encloses the duct system. In some aspects, virtually all of the cells of the glands can be accessed by a single administration into the duct. In this way, it can be possible to transfect large masses of cells with a single procedure. The nucleic acid of interest can thus also be administered without substantial dilution (it is only diluted by the fluid in the duct system) and without the need to develop organ specific targeting signals. In contrast, intravenous administration necessarily greatly dilutes the material and requires that it be targeted to the organ of interest in some fashion. In some aspects, the secretory gland cells are derived from a salivary gland, pineal gland, thyroid gland, adrenal gland, and parathyroid gland. In some aspects, the secretory gland cells are salivary gland cells.

The amount of nucleic acid to transform a sufficient number of secretory gland cells and provide for expression of therapeutic levels of the protein can be assessed using an animal model (e.g., a rodent (mouse or rat) or other mammalian animal model) to assess factors such as the efficiency of transformation, the levels of protein expression achieved, the susceptibility of the targeted secretory gland cells to transformation, and the amounts of vector and/or nucleic acid required to transform secretory gland cells.

The precise amount of vector and/or nucleic acid administered will vary greatly according to a number of factors including the susceptibility of the target cells to transformation, the size and weight of the subject, the levels of protein expression desired, and the condition to be treated.

In some aspects, a viral vectors (e.g., AAV vector) or AAV particles of the present disclosure can be administered to secretory organ (e.g., secretory gland) by intraductal injection. In some aspects, the secretory organ is selected from lymph node, gall bladder, thymus, hypothalamus, stomach, intestine, liver, pancreas, kidney, skin and/or secretory gland. In some aspects, the secretory organ is selected from heart, bone, muscle, skin, and/or adipose tissue. In some aspects, the delivery is by intraductal injection to the salivary gland.

In some aspects, a viral vectors (e.g., AAV vector) or AAV particles of the present disclosure (is administered by direct injection to the secretory organ. In some aspects, the secretory organ is selected from lymph node, gall bladder, thymus, hypothalamus, stomach, intestine, liver, pancreas, kidney, skin and/or secretory gland. In some aspects, the secretory organ is selected from heart, bone, muscle, skin, and/or adipose tissue.

In some aspects, the gene therapy composition or viral vectors (e.g., AAV vector) or AAV particles disclosed herein is administered intraductally, by direct injection to the secretory organ, or both. In some aspects, the gene therapy composition or AAV particle disclosed herein is administered intraductally, by direct injection to the secretory gland, or both. In some aspects, the gene therapy composition or AAV particle disclosed herein is administered intraductally, by direct injection to the salivary gland, or both.

In some aspects, a viral vectors (e.g., AAV vector) or AAV particles of the present disclosure can be administered by intramuscular injection.

In some aspects, a viral vectors (e.g., AAV vector) or AAV particles of the present disclosure can be administered intravenously.

In some aspects, a viral vectors (e.g., AAV vector) or AAV particles of the present disclosure can be administered by intradermal injection.

The viral vectors (e.g., AAV vector) or AAV particles disclosed herein can be administered in any suitable form, either as a liquid solution or suspension, as a solid form suitable for liquid solution or suspension in a liquid solution.

### VIII. Kits

The present disclosure also provides kits, or products of manufacture, comprising (i) the viral vectors (e.g., AAV vector) or AAV particles of the present disclosure, or a pharmaceutical composition of the present disclosure, and (ii) optionally instructions for use (e.g., a package insert with instructions to perform any of the methods described herein).

In some aspects, the kit or product of manufacture comprises (i) comprising the viral vectors (e.g., AAV vector) or AAV particles of the present disclosure or a pharmaceutical composition of the present disclosure, (ii) optionally, an additional therapeutic agent, and (iii) optionally, instructions for use (e.g., a package insert with instructions to perform any of the methods described herein are also contemplated).

In some aspects, a kit or product of manufacture of the present disclosure comprises at least one construct as described herein.

One skilled in the art will readily recognize that viral vectors (e.g., AAV vector) or AAV particles, polynucleotides, and pharmaceutical compositions of the present disclosure, or combinations thereof, can be readily incorporated into one of the established kit formats which are well known in the art.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

The following examples are offered by way of illustration and not by way of limitation.

### EXAMPLES

### Example 1: Nucleic Acid Sequence that Modulates a Toll-Like Receptor

In the present example, a nucleic acid sequence that modulates a toll-like receptor is provided, which sequence can be incorporated into the viral vector constructs, compositions, and methods described herein.

The nucleic acid sequence is as follows:

(SEQ ID NO: 30)
TTAGGGTTAGGGTTAGGGTTAGGGTTAGGGTTAGGGTTAGGGTTAGGG
TTAGGGTTAGGGTTAGGGTTAGGG

The nucleic acid sequence that targets a TLR, SEQ ID NO: 30, can be used to target TLR9.

The nucleic acid sequence can be used as a part of an ITR sequence or near an ITR sequence, e.g. an ITR near a polyA tail, and the flipped sequence, which sequence is as follows, can be used as or near the ITR near the promoter:

(SEQ ID NO: 29)
CCCTAACCCTAACCCTAACCCTAACCCTAACCCTAACCCTAACCCTAA
CCCTAACCCTAACCCTAACCCTAA.

### Example 2: Nucleic Acid Sequence that Modulates a Toll-Like Receptor

In the present example, a nucleic acid sequence that modulates a toll-like receptor is provided, which sequence can be incorporated into the viral vector constructs, compositions, and methods described herein.

The nucleic acid sequence is as follows:

(SEQ ID NO: 32)
TTAGGGTTAGGGTTAGGGTTAGGGTCCTGGCGGGGAAGTTGCTCCTGG
AGGGGTTGTCCTGGATGGGAACTTACCGCTGCATCCTGGAGGGGAAGT

The nucleic acid sequence that targets a TLR, SEQ ID NO: 32, can be used to target TLR7, TLR8, and TLR9.

The nucleic acid sequence can be used as a part of an ITR sequence or near an ITR sequence, e.g. an ITR near a polyA tail, and the flipped sequence, which sequence is as follows, can be used as or near the ITR near the promoter:

(SEQ ID NO: 31)
ACTTCCCCTCCAGGATGCAGCGGTAAGTTCCCATCCAGGACAACCCCT
CCAGGAGCAACTTCCCCGCCAGGACCCTAACCCTAACCCTAACCCTAA.

### Example 3: Nucleic Acid Sequence that Modulates a Toll-Like Receptor

In the present example, a nucleic acid sequence that modulates a toll-like receptor is provided, which sequence can be incorporated into the viral vector constructs, compositions, and methods described herein.

The nucleic acid sequence is as follows:

(SEQ ID NO: 34)
TTAGGGTTAGGGTTAGGGTTAGGGCCTGGATGGGAACTTACCGCTGCATC
CTGGAGGGGAAGTTAATATCCTGGAGGGGAAGTCCTATCCTGGAGGGGAA
GCCTTGGATGGGAA.

The nucleic acid sequence that targets a TLR, SEQ ID NO: 34, can be used to target TLR9.

The nucleic acid sequence can be used as a part of an ITR sequence or near an ITR sequence, e.g. an ITR near a polyA tail, and the flipped sequence, which sequence is as follows, can be used as or near the ITR near the promoter:

(SEQ ID NO: 33)
TTCCCATCCAAGGCTTCCCCTCCAGGATAGGACTTCCCCTCCAGGATAT
TAACTTCCCCTCCAGGATGCAGCGGTAAGTTCCCATCCAGGCCCTAACC
CTAACCCTAACCCTAA.

### Example 4: Nucleic Acid Sequence that Modulates a Toll-Like Receptor

In the present example, a nucleic acid sequence that modulates a toll-like receptor is provided, which sequence can be incorporated into the viral vector constructs, compositions, and methods described herein.

The nucleic acid sequence is as follows:

(SEQ ID NO: 36)
TCCTGGCGGGGAAGTTGCTCCTGGAGGGGTTGTTCGTCGTTCTGTGGC
GCGCACCCACGGCCTG.

The nucleic acid sequence that targets a TLR, SEQ ID NO: 36, can be used to target TLR3, TLR7, TLR8, and TLR9.

The nucleic acid sequence can be used as a part of an ITR sequence or near an ITR sequence, e.g. an ITR near a polyA tail, and the flipped sequence, which sequence is as follows, can be used as or near the ITR near the promoter:

(SEQ ID NO: 35)
CAGGCCGTGGGTGCGCGCCACAGAACGACGAACAACCCCTCCAGGAG
CAACTTCCCCGCCAGGA.

### Example 5: Nucleic Acid Sequence that Modulates a Toll-Like Receptor

In the present example, a nucleic acid sequence that modulates a toll-like receptor is provided, which sequence can be incorporated into the viral vector constructs, compositions, and methods described herein.

The nucleic acid sequence is as follows:

(SEQ ID NO: 38)
TTAGGGTTAGGGTTAGGGTTAGGGTGCTCCTGGAGGGGTTGTCCTGGAT
GGGAACTTACCGCTGCATCCTGGAGGGGAAGTTAATATCCTGGAGGGGA
AGTCCTATCCTGGAGGGGAAGCCTTGGATGGGAATCGTCGTTCTG.

The nucleic acid sequence that targets a TLR, SEQ ID NO: 38, can be used to target TLR7 and TLR9.

The nucleic acid sequence can be used as a part of an ITR sequence or near an ITR sequence, e.g. an ITR near a polyA tail, and the flipped sequence, which sequence is as follows, can be used as or near the ITR near the promoter:

(SEQ ID NO: 37)
CAGAACGACGATTCCCATCCAAGGCTTCCCCTCCAGGATAGGACTTCCC
CTCCAGGATATTAACTTCCCCTCCAGGATGCAGCGGTAAGTTCCCATCC
AGGACAACCCCTCCAGGAGCACCCTAACCCTAACCCTAACCCTAA.

### Example 6

The activity of nucleic acid sequences in modulating TLR9 signaling was tested using the HEK-Dual™ hTLR9 (NF/IL8) Cell system from InvivoGen (USA). HEK-Dual™ TLR9 cells stably express the human TLR9 gene and have TLR3 and TLRS and TNFR genes knocked out allowing the study of human TLR9 without interference from other TLRs. HEK-Dual™ TLR9 cells respond to low concentrations of TLR9 agonists, such as the class B CpG oligonucleotide CpG ODN2006 (5′-TCGTCGTTTTGTCGTTTTGTCGTT-3′, SEQ ID NO: 56), but the cells do not respond to other TLR agonists or to the cytokine TNF-α.

The oligonucleotides tested with HEK-Dual™ TLR9 cells were oligonucleotides (ODNs) comprising a TLR9-stimulatory ODN2006 sequence combined with a test nucleic acid sequence comprising four copies of TTAGGG (SEQ ID NO: 3) (ODN2006-TTAGGG, SEQ ID NO: 50) or a TLR9-stimulatory ODN2006 sequence combined with a test nucleic acid sequence comprising four copies of TTCGCG (ODN2006-TTCGCG, SEQ ID NO: 51). Control oligonucleotides used contained either no TLR-stimulatory sequences (Control-Control, SEQ ID NO: 53) or a 5′ TLR9-stimulatory ODN2006 sequence combined with a 3′ control sequence (ODN2006-Control, SEQ ID NO: 52). The sequences related to the ODNs in this example are shown in Table 4.

TABLE 4

| SEQ | | |
|---|---|---|
| ID NO. | Oligo_ID | Sequence |
| 50 | ODN2006- | TCGTCGTTTTGTCGTTTTGTCGTTTTAGGGTTAGG |
| | TTAGGG | GTTAGGGTTAGGG |
| 51 | ODN2006- | TCGTCGTTTTGTCGTTTTGTCGTTTTCGCGTTCGC |
| | TTCGCG | GTTCGCGTTCGCG |
| 52 | ODN2006- | TCGTCGTTTTGTCGTTTTGTCGTTTTATTATTATT |
| | Control | ATTATTATTATTA |
| 53 | Control- | TTATTATTATTATTATTATTATTATTATTATTATT |
| | Control | ATTATTATTATTA |
| 54 | TTCGCG | TTCGCG |
| 55 | TTAGGG | TTAGGG |
| 56 | ODN2006 | TCGTCGTTTTGTCGTTTTGTCGTT |

HEK-Dual™ TLR9 cells were incubated with the ODNs.

Thereafter, supernatant was removed and secreted embryonic alkaline phosphatase (SEAP) activity was measured.

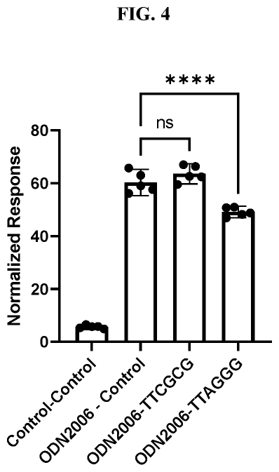

The results showed SEAP activity induced by ODN-2006 following incubation with HEK-Dual™ TLR9 cells was significantly reduced in the presence of the TTAGGG repeat (ODN2006-TTAGGG, SEQ ID NO: 50) (FIG. 4) compared with control oligonucleotides ODN2006-Control (SEQ ID NO: 52) and ODN2006-TTCGCG (SEQ ID NO: 51). These data indicate that an oligonucleotide comprising copies of TTAGGG (SEQ ID NO: 55), e.g., 4 tandem repeats such as SEQ ID NO: 3 significantly reduces TLR9 activation.

### Example 7

The activity of vector constructs and/or portions thereof in modulating TLR9 signaling will be tested using methods similar to those described in Example 6.

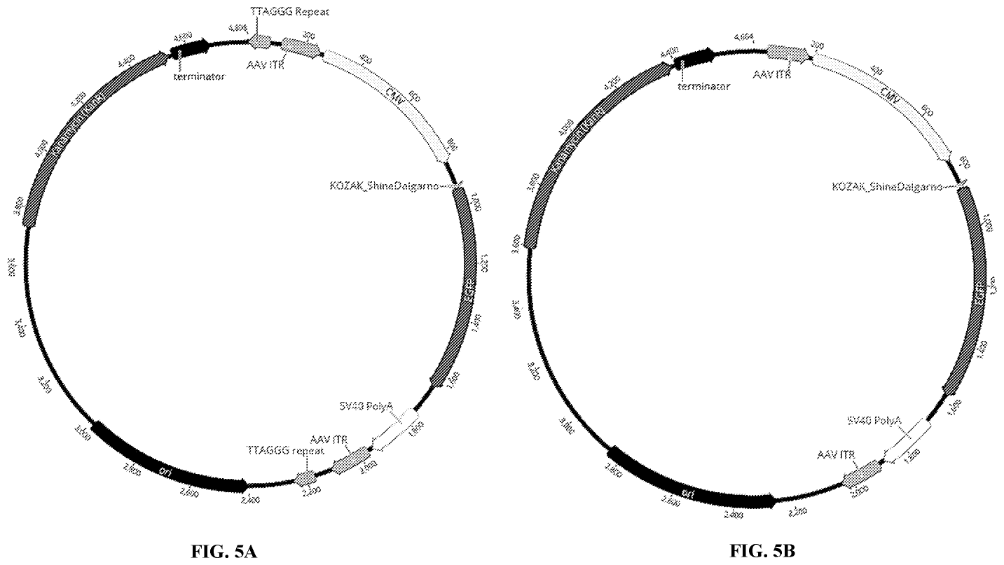

In short, AAV vector construct(s) will be tested with HEK-Dual™ TLR9 cells. The vector will include an expression cassette with flanking AAV ITRs and a backbone (pSF-AAV-ITR-CMV-EGFP-ITR-KanR) where the backbone is modified to include at least four (e.g., twelve (12)) repeats of TTAGGG (SEQ ID NO: 55) outside of the 5′ and the 3′ ends of the ITRs (FIG. 5A). Additionally, a control vector without the TTAGGG (SEQ ID NO: 55) repeats (FIG. 5B) will be used for comparison. The expression cassettes for the test and control vectors will include at least a promoter (e.g. CMV), gene of interest (e.g., EGFP) and polyA (e.g., SV40 PolyA). The length of the expression cassette will be less than the length of a wildtype AAV genome (˜4.7 kb) in order to increase the frequency of packaging of the backbone (e.g. ˜2.0 kb)

HEK-Dual™ TLR9 cells will be transduced with the vector constructs. Thereafter, supernatant will be removed and secreted embryonic alkaline phosphatase (SEAP) activity will be measured to assess suppression of TLR9 activity.

What is claimed is:

1. A vector construct comprising: (a) a polynucleotide comprising a promoter operably linked to a nucleic acid of interest; (b) a first terminal repeat and a second terminal repeat; and (c) a backbone polynucleotide comprising a nucleic acid sequence that modulates a Toll-like receptor (TLR).

2. The vector construct of claim 1, wherein the first and second terminal repeats are inverted terminal repeats or long terminal repeats.

3. The vector construct of any of the foregoing claims, wherein the backbone polynucleotide comprises two or more copies of the nucleic acid sequence that modulates a TLR.

4. The vector construct of any of the foregoing claims, wherein the backbone polynucleotide comprises between 2 to 500 copies, between 2 to 200 copies, between 2 to 150 copies, between 2 to 100 copies, between 2 to 50 copies, between 2 to 40 copies, between 2 to 30 copies, between 2 to 25 copies, between 2 to 20 copies, between, 2 to 15 copies, or between 2 to 10 copies of the nucleic acid sequence that modulates the TLR.

5. The vector construct of any of the foregoing claims, wherein the backbone polynucleotide comprises a linker positioned between the two or more copies of the nucleic acid sequence that modulates the TLR.

6. The vector construct of claim 5, wherein the linker is 3 to 25 nucleotides in length.

7. The vector construct of claim 5 or 6, wherein the two or more copies of the nucleic acid sequence that modulates the TLR are positioned in tandem with the linker in between.

8. The vector construct of any of the foregoing claims, wherein the nucleic acid sequence that modulates the TLR is positioned within 500 nucleotides or less, 450 nucleotides or less, 400 nucleotides or less, 350 nucleotides or less, 300 nucleotides or less, 250 nucleotides or less, 200 nucleotides or less, 150 nucleotides or less, 100 nucleotides or less, or 50 nucleotides or less from the first terminal repeat or the second terminal repeat.

9. The vector construct of any of the foregoing claims, wherein the TLR comprise a TLR3, a TLR4, a TLR7, a TLR8, a TLR9, or any combination thereof.

10. The vector construct of any of the foregoing claims, wherein the TLR comprises TLR9.

11. The vector construct of any of the foregoing claims, wherein the backbone polynucleotide comprises two or more copies of the nucleic acid sequences that modulates TLR9.

12. The vector construct of any of the foregoing claims, wherein the nucleic acid sequence that modulates the TLR comprises a sequence selected from any of SEQ ID NOs: 1-38, or any combination thereof.

13. The vector construct of any of the foregoing claims, wherein the backbone polynucleotide is at least 4000 nucleic acids in length.

14. The vector construct of any of the foregoing claims, wherein the backbone polynucleotide is about 4000 to about 8000 nucleic acids in length or about 5000 to about 7000 nucleic acids in length.

15. The vector construct of any of the foregoing claims, wherein the nucleic acid sequence that modulates the TLR comprises about 0.5% to about 10%, about 0.5% to about 5%, about 0.5% to about 4%, about 0.5% to about 3%, about 0.5% to about 2%, or about 0.5% to about 1% of the total nucleic acid sequence of the backbone polynucleotide.

16. The vector construct of any of the foregoing claims, wherein the nucleic acid sequence that modulates the TLR is capable of inhibiting or reducing a TLR inflammatory response.

17. The vector construct of any of claims 1-15, wherein the nucleic acid sequence that modulates the TLR is capable of activating or increasing an inflammatory response.

18. The vector construct of any of the prior claims, wherein the first terminal repeat is an ITR comprising about 75 to about 175 nucleotides in length.

19. The vector construct of any of the prior claims, wherein the second terminal repeat is an ITR comprising about 75 to about 175 nucleotides in length.

20. The vector construct of any of the prior claims, wherein the first terminal repeat and/or the second terminal repeat is an ITR from an AAV serotype selected from AAV1, AAV2, AAV3a, AAV3b, AAV4, AAV5, AAV6, AAV7, AAV8, AAV9, AAV10, AAV11, or AAV13; or any functional fragment thereof.

21. A composition comprising the vector construct of any of the foregoing claims.

22. A method for packaging the nucleic acid of interest and the nucleic acid sequence that modulates a TLR in an AAV capsid, comprising transfecting a cell in vitro with a vector construct of any of claim 1-20 and one or more AAV packaging genes, wherein the nucleic acid of interest and the nucleic acid sequence that modulates the TLR are packaged in the AAV capsid.

23. The method of claim 22, wherein the AAV capsid is an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

24. The method of claim 22 or 23, wherein the one or more AAV packaging genes comprises Rep/Cap genes and adenovirus genes.

25. An AAV particle produced by the method of any one of claims 22-24.

26. The AAV particle of claim 25, wherein the AAV particle comprises about 0.01% to about 2% of the polynucleotide backbone sequence from the vector construct.

27. The AAV particle of claim 25 or 26, wherein the AAV particle comprises about 0.01% to about 1.5% of the polynucleotide backbone sequence from the vector construct.

28. The AAV particle of any of claims 25-27, wherein the AAV particle comprises about 0.01% to about 1% of the polynucleotide backbone sequence from the vector construct.

29. The AAV particle of any of claims 25-28, wherein the TLR comprise a TLR3, a TLR4, a TLR7, a TLR8, a TLR9, or any combination thereof.

30. The AAV particle of any of claims 25-29, wherein the TLR comprises TLR9.

31. The AAV particle of any of claims 25-30, wherein the backbone polynucleotide comprises two or more copies of the nucleic acid sequences that modulates TLR9.

32. The AAV particle of any of claims 25-31, wherein the nucleic acid sequence that modulates the TLR comprises a sequence selected from any of SEQ ID NOs: 1-38, or any combination thereof.

33. A method of modulating an immune response in a subject, comprising administering to said subject an effective amount of the AAV particle of any of claims 25-32.

34. The method of claim 33, wherein the nucleic acid sequence that modulates the TLR is capable of inhibiting a TLR inflammatory response.

35. The method of claim 34, which reduces the subject's immune response to a gene therapy comprising administration of the viral particle to the subject.

36. The method of claim 33, wherein the nucleic acid sequence that modulates the TLR is capable of activating an inflammatory response.

37. The method of claim 36, which enhances the subject's immune response to a tumor.

38. A method of reducing immunogenicity of an AAV particle comprising packaging a portion of a backbone polynucleotide comprising a nucleic acid sequence that modulates a Toll-like receptor (TLR) into the AAV particle, whereby the AAV particle causes a reduced inflammatory response in a host as compared to an AAV particle that does not contain the portion of a backbone comprising the nucleic acid sequence that modulates the TLR.

39. A method of enhancing immunogenicity of an AAV particle comprising packaging a portion of a backbone polynucleotide comprising a nucleic acid sequence that modulates a Toll-like receptor (TLR) into the AAV particle, whereby the AAV particle causes an enhanced inflammatory response in a host as compared to an AAV particle that does not contain the portion of a backbone comprising the nucleic acid sequence that modulates the TLR.

40. The method of claim 38 or 39, wherein the AAV particle comprises an AAV capsid having an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

41. The method of any of claim 38-40, wherein the AAV particle comprises about 0.01% to about 2% of the polynucleotide backbone sequence.

42. The method of any of claim 38-41, wherein the AAV particle comprises about 0.01% to about 1% of the polynucleotide backbone sequence.

43. The method of any of claim 38-42, wherein the TLR comprise a TLR3, a TLR4, a TLR7, a TLR8, a TLR9, or any combination thereof.

44. The method of any of claim 38-43, wherein the TLR comprises TLR9.

45. The method of any of claim 38-44, wherein the backbone polynucleotide comprises two or more copies of the nucleic acid sequences that modulates TLR9.

46. The method of any of claim 38-45, wherein the nucleic acid sequence that modulates the TLR comprises a sequence selected from any of SEQ ID NOs: 1-38, 54, 55, or any combination thereof.

47. The vector construct of any one of claims 1-11, AAV particle of any one of claims 25-32, or the method of any one of claims 12-24 and 33-46, wherein the nucleic acid sequence that modulates TLR comprises two or more tandem repeats of the sequence of SEQ ID NO: 55.

48. The vector construct of any one of claims 1-11, AAV particle of any one of claims 25-32, or the method of any one of claims 12-24 and 33-46, wherein the nucleic acid sequence that modulates TLR comprises the sequence of SEQ ID NO: 3.

49. The vector construct of claim 47 or 48, wherein the nucleic acid sequence that modulates TLR comprises between one and 500 copies of the sequence of SEQ ID NO: 3.

50. A vector construct, AAV particle, or the method of any one of claims 1-49, wherein the polynucleotide comprising the promoter operably linked to a nucleic acid of interest is located between the first and second terminal repeat and measures in length less than a viral genome.

51. The vector construct of claim 50, wherein the viral genome is a single-stranded AAV viral genome.

52. The vector construct of claim 51, wherein the polynucleotide comprising the promoter operably linked to a nucleic acid of interest measures less than 4700 nucleotides.

53. The vector construct of claim 50, wherein the viral genome a self-complementary AAV genome.

54. The vector construct of claim 53, wherein the polynucleotide comprising the promoter operably linked to a nucleic acid of interest is self-complementary and measures less than 2300 nucleotides.

55. The vector construct of any one of claims 50 to 54, wherein one or more of the nucleic acid sequence that modulates a TLR is adjacent to a first or a second AAV inverted terminal repeat.

56. The vector construct of any of claims 49-55, wherein the backbone polynucleotide comprises between 2 to 500 copies, between 2 to 200 copies, between 2 to 150 copies, between 2 to 100 copies, between 2 to 50 copies, between 2 to 40 copies, between 2 to 30 copies, between 2 to 25 copies, between 2 to 20 copies, between, 2 to 15 copies, or between 2 to 10 copies of the nucleic acid sequence that modulates the TLR.

57. A composition comprising the vector construct or AAV particle of any of the claim 1-11, 24-32, or 47-56.

58. The AAV particle, composition or method of any one of claims 25-57, wherein the AAV particle comprises about 0.01% to about 2% of the polynucleotide backbone sequence from the vector construct.

59. The AAV particle, composition or method of claim 67, wherein the AAV particle comprises about 0.01% to about 10% of the polynucleotide backbone sequence from the vector construct.


SEQUENCE LISTING (56 sequences)

SEQ ID NO: 1 - DNA, 15 bp, Artificial Sequence
  polynucleotide that modulates at least TLR9
tcctggaggggaagt

SEQ ID NO: 2 - DNA, 13 bp, Artificial Sequence
  polynucleotide that modulates at least TLR9
ccttggatgggaa

SEQ ID NO: 3 - DNA, 24 bp, Artificial Sequence
  polynucleotide that modulates at least TLR9
ttagggttagggttagggttaggg

SEQ ID NO: 4 - DNA, 15 bp, Artificial Sequence
  polynucleotdie that modulates at least TLR9
tcctggatgggaagt

SEQ ID NO: 5 - DNA, 24 bp, Artificial Sequence
  polynucleotide that moldulates at least TLR9
ttcccatccaggcctggatgggaa

SEQ ID NO: 6 - DNA, 24 bp, Artificial Sequence
  polynucleotide that modulates at least TLR9
cttaccgctgcacctggatgggaa

SEQ ID NO: 7 - DNA, 20 bp, Artificial Sequence
  polynucleotide that modulates at least TLR9
gggggggggggggggggggg

SEQ ID NO: 8 - DNA, 22 bp, Artificial Sequence
  polynucleotide that modulates at least TLR9
tgactgtgaaggttagagatga

SEQ ID NO: 9 - DNA, 15 bp, Artificial Sequence
  polynucleotide that modulates at least TLR9
tcctggaggggttgt

SEQ ID NO: 10 - DNA, 10 bp, Artificial Sequence
  polynucleotide that modulates at least TLR9
cctggcgggg

SEQ ID NO: 11 - DNA, 24 bp, Artificial Sequence
  polynucleotide that modulates at least TLR9
cctggatgggaattcccatccagg

SEQ ID NO: 12 - DNA, 24 bp, Artificial Sequence
  polynucleotide that modulates at least TLR9
cctggatgggaacttaccgctgca

SEQ ID NO: 13 - DNA, 21 bp, Artificial Sequence
  polynucleotide that modulates at least TLR9
ctcctattgggggtttcctat

SEQ ID NO: 14 - DNA, 19 bp, Artificial Sequence
  polynucleotide that modulates at least TLR9
tcgtatcctggaggggaag

SEQ ID NO: 15 - DNA, 18 bp, Artificial Sequence
  polynucleotide that modulates at least TLR9
cctatcctggaggggaag

SEQ ID NO: 16 - DNA, 19 bp, Artificial Sequence
  polynucleotide that modulates at least TLR9
taatatcctggaggggaag

SEQ ID NO: 17 - DNA, 19 bp, Artificial Sequence
  polynucleotide that modulates at least TLR9
tcctatcctggaggggaag

SEQ ID NO: 18 - DNA, 15 bp, Artificial Sequence
  polynucleotide that modulates at least TLR7, TLR8, and TLR9
tcctggcggggaagt

SEQ ID NO: 19 - DNA, 8 bp, Artificial Sequence
  polynucleotide that modulates at least TLR 7 and TLR9
tcctggcg

SEQ ID NO: 20 - DNA, 11 bp, Artificial Sequence
  polynucleotide that modulates at least TLR 7 and TLR9
tcgtcgttctg

SEQ ID NO: 21 - DNA, 23 bp, Artificial Sequence
  polynucleotide that modulates at least TLR7, TLR8, and TLR9
  misc_feature (12)..(12): n can be A, T, G, or C; and n = 0 or more nucleotides in length
ctgtcgttctcnctcttgctgtc

SEQ ID NO: 22 - DNA, 23 bp, Artificial Sequence
  polynucleotide that modulates at least TLR7, and TLR9
  misc_feature (12)..(12): n can be A, T, G, or C; and n = 0 or more nucleotides in length
tcgtcgttctgngtcttgctgct

SEQ ID NO: 23 - DNA, 20 bp, Artificial Sequence
  polynucleotide that modulates at least TLR3, TLR7, and TLR9
tggcgcgcacccacggcctg

SEQ ID NO: 24 - DNA, 20 bp, Artificial Sequence
  polynucleotide that modulates at least TLR9
cttcgcgttagcgttgagtg

SEQ ID NO: 25 - DNA, 20 bp, Artificial Sequence
  polynucleotide that modulates at least TLR9
ttgcgcgccctccatcgagg

SEQ ID NO: 26 - DNA, 18 bp, Artificial Sequence
  polynucleotide that modulates at least TLR7 and TLR9
tgctcctggaggggttgt

SEQ ID NO: 27 - DNA, 20 bp, Artificial Sequence
  polynucleotide that modulates at least TLR7
tgcttgcaagcttgcaagca

SEQ ID NO: 28 - DNA, 76 bp, Artificial Sequence
  polynucleotide that modulates at least TLR4
gcggatgaagactggtgtgccaataaaccatatcgccgcgttagcatgtactcggttggccctaaatacgagcaac

SEQ ID NO: 29 - DNA, 72 bp, Artificial Sequence
  TERMINAL REPEAT SEQUENCE NEAR PROMOTER
ccctaaccctaaccctaaccctaaccctaaccctaaccctaaccctaaccctaaccctaaccctaaccctaa

SEQ ID NO: 30 - DNA, 72 bp, Artificial Sequence
  TERMINAL REPEAT SEQUENCE NEAR POLYA
ttagggttagggttagggttagggttagggttagggttagggttagggttagggttagggttagggttaggg

SEQ ID NO: 31 - DNA, 96 bp, Artificial Sequence
  TERMINAL REPEAT SEQUENCE NEAR PROMOTER
acttcccctccaggatgcagcggtaagttcccatccaggacaacccctccaggagcaacttccccgccaggaccctaaccctaaccctaaccctaa

SEQ ID NO: 32 - DNA, 96 bp, Artificial Sequence
  TERMINAL REPEAT SEQUENCE NEAR POLYA
ttagggttagggttagggttagggtcctggcggggaagttgctcctggaggggttgtcctggatgggaacttaccgctgcatcctggaggggaagt

SEQ ID NO: 33 - DNA, 114 bp, Artificial Sequence
  TERMINAL REPEAT SEQUENCE NEAR PROMOTER
ttcccatccaaggcttcccctccaggataggacttcccctccaggatattaacttcccctccaggatgcagcggtaagttcccatccaggccctaaccctaaccctaaccctaa

SEQ ID NO: 34 - DNA, 114 bp, Artificial Sequence
  TERMINAL REPEAT SEQUENCE NEAR POLYA
ttagggttagggttagggttagggcctggatgggaacttaccgctgcatcctggaggggaagttaatatcctggaggggaagtcctatcctggaggggaagccttggatgggaa

SEQ ID NO: 35 - DNA, 64 bp, Artificial Sequence
  TERMINAL REPEAT SEQUENCE NEAR PROMOTER
caggccgtgggtgcgcgccacagaacgacgaacaacccctccaggagcaacttccccgccagga

SEQ ID NO: 36 - DNA, 64 bp, Artificial Sequence
  TERMINAL REPEAT SEQUENCE NEAR POLYA
tcctggcggggaagttgctcctggaggggttgttcgtcgttctgtggcgcgcacccacggcctg

SEQ ID NO: 37 - DNA, 143 bp, Artificial Sequence
  TERMINAL REPEAT SEQUENCE NEAR PROMOTER
cagaacgacgattcccatccaaggcttcccctccaggataggacttcccctccaggatattaacttcccctccaggatgcagcggtaagttcccatccaggacaacccctccaggagcaccctaaccctaaccctaaccctaa

SEQ ID NO: 38 - DNA, 143 bp, Artificial Sequence
  TERMINAL REPEAT SEQUENCE NEAR POLYA
ttagggttagggttagggttagggtgctcctggaggggttgtcctggatgggaacttaccgctgcatcctggaggggaagttaatatcctggaggggaagtcctatcctggaggggaagccttggatgggaatcgtcgttctg

SEQ ID NO: 39 - DNA, 5 bp, Artificial Sequence
  Linker
aaaaa

SEQ ID NO: 40 - DNA, 20 bp, Artificial Sequence
  Linker
attgatattcgttggttttg

SEQ ID NO: 41 - DNA, 20 bp, Artificial Sequence
  Linker
aattgatattcgttggtttt

SEQ ID NO: 42 - DNA, 21 bp, Artificial Sequence
  Linker
ttggaagaaattgatattcgt

SEQ ID NO: 43 - DNA, 20 bp, Artificial Sequence
  Linker
gagtgaattatgaatgaaat

SEQ ID NO: 44 - DNA, 20 bp, Artificial Sequence
  Linker
aaagcttttgaggttggtgg

SEQ ID NO: 45 - DNA, 20 bp, Artificial Sequence
  Linker
tcatctttaccggtcatgga

SEQ ID NO: 46 - DNA, 21 bp, Artificial Sequence
  Linker
tctttaccggtcatgggacgg

SEQ ID NO: 47 - protein, 5 aa, Artificial Sequence
  furin cleavage site consensus sequence
  MISC_FEATURE (2)..(2): Xaa can be any amino acid
RXKRR

SEQ ID NO: 48 - RNA, 9 nt, Artificial Sequence
  Kozak consensus sequence
  misc_feature (3)..(3): R can be A or G
ccrccaugg

SEQ ID NO: 49 - DNA, 16 bp, Artificial Sequence
  inverted repeat sequence
  misc_feature (6)..(12): n can be A, T, G, or C
ttacgnnnnnncgtaa

SEQ ID NO: 50 - DNA, 48 bp, Artificial Sequence
  ODN2006-TTAGGG
tcgtcgttttgtcgttttgtcgttttagggttagggttagggttaggg

SEQ ID NO: 51 - DNA, 48 bp, Artificial Sequence
  ODN2006-TTCGCG
tcgtcgttttgtcgttttgtcgttttcgcgttcgcgttcgcgttcgcg

SEQ ID NO: 52 - DNA, 48 bp, Artificial Sequence
  ODN2006-Control
tcgtcgttttgtcgttttgtcgttttattattattattattattatta

SEQ ID NO: 53 - DNA, 48 bp, Artificial Sequence
  Control-Control
ttattattattattattattattattattattattattattattatta

SEQ ID NO: 54 - DNA, 6 bp, Artificial Sequence
  TTCGCG
ttcgcg

SEQ ID NO: 55 - DNA, 6 bp, Artificial Sequence
  TTAGGG
ttaggg

SEQ ID NO: 56 - DNA, 24 bp, Artificial Sequence
  ODN2006
tcgtcgttttgtcgttttgtcgtt